SEQUENCE LISTING (14 sequences)

SEQ ID NO: 1 - protein, 2710 aa, Clostridium difficile
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNIIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALISYKIINGKHFYFNNDGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 2 - protein, 2862 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEAMSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 3 - protein, 879 aa, Clostridium difficile
GLININNSLFYFDPIEFNLVTGWQTINGKKYYFDINTGAALISYKIINGKHFYFNNDGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 4 - protein, 533 aa, Clostridium difficile
GLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 5 - protein, 449 aa, synthetic construct
  NON_STD 1: note = Residue 1 is lipidated
CSSFVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYGLEHHHHHH

SEQ ID NO: 6 - protein, 528 aa, Synthetic construct
  NON_STD 1: note = Residue 1 is lipidated
CSSFNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISELEHHHHHH

SEQ ID NO: 7 - protein, 961 aa, Synthetic construct
  NON_STD 1: note = Residue 1 is lipidated
CSSFVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYGRSMHNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 8 - protein, 607 aa, Clostridium difficile
MKKKAALATLAMLPLGVVNAHADGDIGIVTINYLNVRNEPTAESSIAFVAKKDDKVLIKDSSNGWYKIKAESGQEGWASSKYIAKSNSDSLRTSTNKEKQVISNSLNMRNGAGTSYRVITVLKKGQKVEVISESNGWSKIKYDGRLGYVSSSYLGDVSNSTNKSKTKQVNTTSLNVRSGPNTSYGLLGKLPKGSKVEVISESNGWSKIKYNGKDAYISSMYLSDVSQSNSDNSSQSNDKKNTDKVVNTASLNVRSGPGSTYSKLGKVYKGSKVTVLSESSGWAKINFNNKEAFVVGNYLSTSADTSNNNSNSNSDNSSNSNGNNSSSSGQVNGMSGISGAKIDYKSLSYTLESHISKQVEKAASGGNVIAPSNRKSTPSPEFSTFSAQRTSSFVNASSSDIEYYLNPKNFTNTTKGMMQFLKINSYRDGISESSLNSYLNGLSSSVFKNQGAAFINAAKKYNIDVVYLVSHAMWETAYGKSTLAQGQTLTSYKGQPLSKPVKVYNFFGIGAIDKSANVSGAEAAYSNGWTSVEATIDGSAKWISQNYVNSSKYNQNTIYKMKWNYDYTWHQYATDVNWANGISGIMENLIGLYGGGSSLVFEVPQYK

SEQ ID NO: 9 - protein, 396 aa, Clostridium difficile
MNFNQKRIAASIMATAIIMPTMGNLAYANESEVESVSIESRTITGNAVNFRKGPGTNHESMGKLYKGDKVEYVGKEGSWVKVKYNGNTGYVHGNYVAINSLGSSNESSDTSVKSTKVVTAKGLNFRTGPSTSSSKISTLGYGTEVGYISESNGWSKISSNGRVGYVSSKYLGTSVNDSTNENVENSSNDLVKGTKVVTAKSLNVRTGPGTSHSKIATLSYGTEVGSISESGGWTKVSYGNQTGYVSSQYLAEKGSVDTSIPSYSTNSPSQGADSVISFAKTLLGKPYVWGAEGPNSFDCSGFTQYVMKKSAGVSIPRVSRDQSKYGTYVNRGDLRSGDLVFFDTQGSNNGSVSHVGIYIGNGDMIHASSGSSKKVTISNINSSYYSSRYVNARRVL

SEQ ID NO: 10 - DNA, 22 bp, Synthetic construct
  source 1..22: mol_type = other DNA
tgactgtgaacgttcgagatga

SEQ ID NO: 11 - DNA, 20 bp, Synthetic construct
  source 1..20: mol_type = other DNA
tccatgacgttcctgacgtt

SEQ ID NO: 12 - DNA, 24 bp, Synthetic construct
  source 1..24: mol_type = other DNA
tcgtcgttttgtcgttttgtcgtt

SEQ ID NO: 13 - protein, 11 aa, Synthetic construct
KLKLLLLLKLK

SEQ ID NO: 14 - DNA, 26 bp, Synthetic construct
  source 1..26: mol_type = other DNA
  misc_feature 1..26: standard_name = n=deoxyinosine
ncncncncncncncncncncncncnc

(19) United States
(12) Patent Application Publication
(10) Pub. No.: US 2025/0213667 A1
(43) Pub. Date: Jul. 3, 2025

(54) CLOSTRIDIUM DIFFICILE VACCINE

(71) Applicant: Valneva Austria GmbH, Vienna (AT)

(72) Inventors: Urban Lundberg, Pressbaum (AT); Andreas Meinke, Pressbaum (AT)

(73) Assignee: Valneva Austria GmbH, Vienna (AT)

(21) Appl. No.: 18/867,201

(22) PCT Filed: May 31, 2023
(86) PCT No.: PCT/EP2023/064602

§ 371 Date: Nov. 19, 2024

(30) Foreign Application Priority Data
Jun. 1, 2022 (EP) 22176663.7

(51) Int. Cl. A61K 39/08; A61K 39/00; A61K 39/39; A61P 37/04

(52) U.S. Cl. CPC A61K 39/08; A61K 39/39; A61P 37/04; A61K 2039/55505; A61K 2039/55561; A61K 2039/6018

(57) ABSTRACT

The present invention relates to a lipidated immunogenic Clostridium difficile toxin A and toxin B polypeptide, a pharmaceutical composition comprising the immunogenic Clostridium difficile toxin A and/or toxin B polypeptide for use as a medicament, particularly a vaccine and/or for use in a method for the prevention or treatment of C. difficile infection and/or C. difficile-associated disease.

### FIELD OF THE INVENTION

The present invention is directed to immunogenic compositions, methods of making vaccines, and methods of vaccine administration. Specifically, the invention relates to Clostridium difficile vaccines comprising (a) a polypeptide comprising a lipidated non-toxic, immunogenic polypeptide fragment of Clostridium difficile Toxin B and (b) optionally further Clostridium difficile antigens.

### BACKGROUND OF THE INVENTION

Clostridium difficile, a multi-drug resistant, spore-forming bacterium on the CDC 2013 Urgent Threats list Antibiotic Resistance Threats in the United States, 2013 (AR Threats Report) https://www.cdc.gov/drugresistance/biggest_threats.html), is a common cause of healthcare-acquired infections occurring principally in older adults taking antibiotic regimens or experiencing prolonged hospital stays. Ironically, although C. difficile infections (CDI) are not yet significantly resistant to antibiotics, most infections are directly related to antibiotic therapy. Thus, CDI is commonly termed antibiotic associated diarrhea (AAD).

Clostridium difficile is recognized as the most important single identifiable cause of nosocomial antibiotic-associated diarrhea and colitis, and CDI has now also emerged in the community in populations previously considered low risk, such as healthy peripartum women, children, antibiotic naive patients, and those with minimal or no recent healthcare exposure (Centers for Disease Control and Prevention (CDC), Severe Clostridium difficile associated disease in populations previously at low risk-four states, MMWR Morb Mortal Wkly Rep., 54: 1201-1205 (2005)).

In recent years, a dramatic increase in the incidence of C. difficile diarrhea has been observed, noted by a marked increase in incidence and severity. A 2015 CDC study found that there were nearly half a million cases of CDI in the United States per year that led to 15,000 deaths. The average cost for a single inpatient case of CDI is >$35,000 and the estimated annual cost burden for the healthcare system exceeds $3 billion.

C. difficile exerts its effects on the gastrointestinal (GI) tract by releasing two toxins that can bind to and damage intestinal epithelium. Toxins A (an enterotoxin) and B (a cytotoxin) contribute differently to the pathophysiology of CDI. Toxin A is associated with the secretion of fluid and generalized inflammation in the GI tract. Toxin B is considered the main determinant of virulence in recurrent CDI and is associated with more severe damage to the colon (Centers for Disease Control and Prevention Healthcare associated infections; https://www.cdc.gov/hai/organisms/cdiff/cdiff clinicians.html).

The potential severity of and damage caused by CDI in combination with its rising incidence renders the subject of prophylaxis a pressing public health concern. Previous research into various toxoid vaccines suggests their promise as preventative measures. Three investigational vaccines have been evaluated in Phase 2/3 clinical trials. Vaccine candidates in advanced clinical development target Toxin A and Toxin B. Recently, Sanofi discontinued development of its C. difficile toxoid A and toxoid B combination vaccine, indicating a toxoid- only prophylactic approach is not sufficient to prevent CDI recurrent disease. Pfizer and Valneva continue to advance their respective non-toxic, immunogenic polypeptide fragment of Clostridium difficile vaccine programs. Very recently, Pfizer published its phase 3 CLOVER Trial for its investigational Clostridioides Difficile Vaccine which indicated a strong potential effect in reducing duration and severity of disease based on secondary endpoints (see https://www.pfizer.com/news/press-release/press-release-detail/phase-3-clover-trial-pfizers-investigational-clostridioides). Furthermore, the FDA has approved Merck's bezlotoxumab (Zinplava™), a monoclonal antibody targeting C. difficile Toxin B, for use in combination with antibiotic therapy for treatment of patients with CDI for the prevention of recurrent CDI, however Zinplava™ is only partially effective in ameliorating the symptoms associated with CDI, is less effective against hypervirulent C. difficile strains, and a decrease in CDI recurrence of only about 40% was observed in patients with CDI.

In view of the increasing incidence of CDI and the absence from the market of an effective vaccine for prevention of CDI, the need remains to discover an effective prophylactic approach for raising an immune response that will be protective against C. difficile infection. Furthermore, there is a need for improved therapeutic vaccines and immunotherapies for treatment of CDI.

### SUMMARY OF THE INVENTION

The present invention provides immunogenic compositions comprising a lipidated Clostridium difficile (hereinafter also referred to as “CD”) toxin B polypeptide for use in the prevention or treatment of CD infection and/or CD associated disease (CDAD) in a subject. Preferably the lipidated polypeptide comprises a CD toxin B cell-binding domain or fragment thereof wherein the lipidated polypeptide lacks the CD toxin A cell-binding domain sequence and optionally comprises a further antigen directed against a CD antigen.

### BRIEF DESCRIPTION OF THE DRAWINGS

FIG. 1. Test of immunogenicity and protection with lipidated constructs.

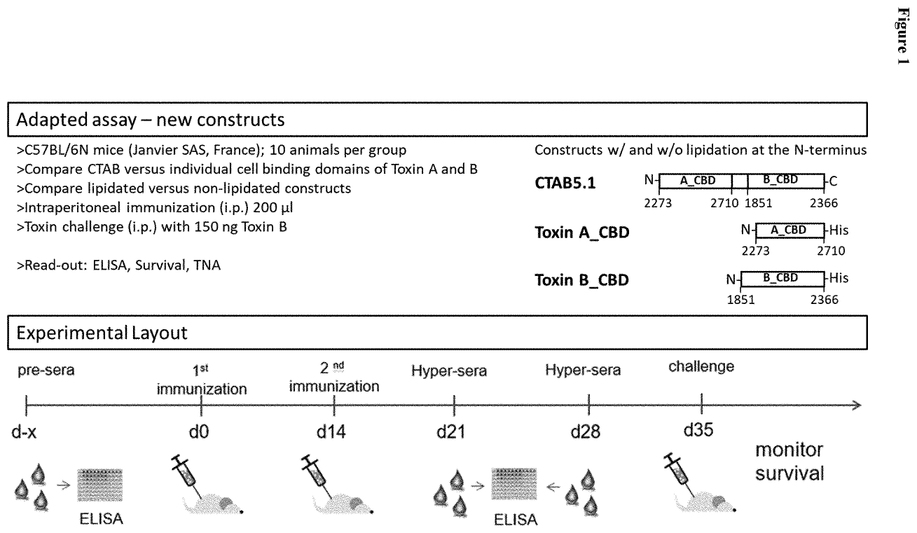

FIG. 2. ELISA GMT IgG titers for lipidated and non-lipidated constructs: comparison of ELISA titers against C-TAB.G5.1 (herein also referred to simply as “CTAB”), toxin B and toxin A for lipidated and non-lipidated CTAB, toxin B cell-binding domain and the combination of toxin A and toxin B cell-binding domains.

FIG. 3. Correlation between anti-toxin B IgG titer and survival of immunized mice after Toxin B challenge: higher IgG titer supports survival and lower titers let to death; particularly, anti-toxin B IgG titer>100,000 protects (with one exception) mice from death.

FIG. 4. Immunogenicity of lipidated and non-lipidated constructs. Statistical significant increased immune response with lipidated constructs (ToxinB_CBD and ToxinA_CBD+ToxinB_CBD). Reduced IgG ELISA response against toxin B when ToxinA_CBD is added, less pronounced for toxin A when ToxinB_CBD is added and for lipidated constructs, effect not seen in CTAB ELISA.

### DETAILED DESCRIPTION OF THE INVENTION

The present invention provides a method of preventing, treating, or alleviating one or more symptoms of a disease, such as CDAD by administering the isolated lipidated polypeptide of the invention to a subject in need thereof. The immunogenic compositions comprising a lipidated CD toxin B polypeptide for use in the prevention or treatment of CD infection and/or CD associated disease (CDAD) may be administered to the subject intramuscularly or by other routes of delivery.

In one embodiment, the present invention provides a method of preventing (or protection against) and/or treating a disease, such as CDAD by administering the isolated, lipidated polypeptide of the inventions or a composition comprising said polypeptide to a subject at risk of CDAD, such as e.g. a subject with the following profile: i) a subject with a weaker immune system such as e.g. an elderly subject (e.g. a subject above 65 years of age) or a subject below 2 years of age; ii) an immunocompromised subject such as e.g. a subject with AIDS; iii) a subject taking or planning to take immunosuppressing drugs; iv) a subject with planned hospitalization or a subject that is in hospital; v) a subject in or expected to go to an intensive care unit (ICU); vi) a subject that is undergoing or is planning to undergo gastrointestinal surgery; vii) a subject that is in or planning to go to a long-term care such as a nursing home; viii) a subject with co-morbidities requiring frequent and/or prolonged antibiotic use; ix) a subject that is a subject with two or more of the above mentioned profiles, such as e.g. an elderly subject that is planning to undergo a gastrointestinal surgery; x) a subject with inflammatory bowel disease; and/or xi) a subject with recurrent CDAD such as e.g. a subject having experienced one or more episodes of CDAD.

In order to provide a lipidated protein in an amount sufficient for commercial use, e.g. as a vaccine to be introduced to the market and used in health care, it has to be produced on large scale. However, upscaling production processes often results in structural changes of the product. When upscaling the production of lipidated CD fusions, it was found that the lipidation profile of said fusions may change depending on the conditions selected during the production in E. coli cells in a fed-batch process (see WO2021/205022, whole content herewith incorporated). Particularly, it has been found that fermenter headspace pressure and pH, optionally in combination with trace elements and anti-foaming agents are of relevance. This particularly applies to any subunit vaccine such as the CD polypeptides described herein. The invention in WO2021/205022 solves this problem and thus it can readily be applied to the polypeptides and composition for use according to the invention.

The present invention provides lipidated polypeptides, wherein the lipidated polypeptides has one to three lipids attached to a glycerol and the N-terminal cysteine of the polypetide, particularly wherein the lipidated polypeptide has one lipid and a glycerol substituted with two lipids attached to the amino group of the N-terminal cysteine and/or particularly wherein the three acyl residues of the lipids are independently selected from C₁₄₋₂₀ alkyl and/or C₁₄₋₂₀ alkenyl, preferably wherein the lipidated polypeptide has the formula (I):

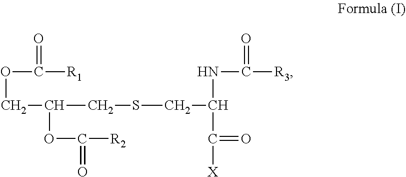

in which R₁, R₂ and/or R₃ are independently selected from C₁₄-C₂₀ alkyl or C₁₄-C₂₀ alkenyl and in which X is an amino acid sequence attached to the cysteine residue.

The present invention also provides the method of production a lipidated polypeptide or protein, which comprises:
a) culturing E co/i cells producing the lipidated protein in a volume of at least 40 L under a defined pressure and a defined pH;
b) harvesting the produced lipidated protein by extraction from E. coli cell culture, with e.g. a detergent, e.g. Triton X-114,
wherein the pressure and the pH are selected to obtain a lipidation profile of the lipidated proteins, in which about 40-60% of the fatty acids are palmitic acid (16:0), about 10 to 20% are mono-unsaturated fatty acids comprising 17 C atoms, about 10 to 20% are mono-unsaturated fatty acids comprising 18 C atoms, about 5 to 20% are mono-unsaturated fatty acids comprising 16 C atoms and about 0 to 10% are other fatty acids, particularly in which about 50% of the fatty acids are palmitic acid (16:0), about 10 to 20% are mono-unsaturated fatty acids comprising 17 C atoms, about 10 to 20% are mono-unsaturated fatty acids comprising 18 C atoms, about 8 to 15% are mono-unsaturated fatty acids comprising 16 C atoms and about 1 to 5% are cyclopropane-comprising fatty acids having 19 C atoms.

Alternatively, the pressure and the pH are selected to obtain an RP-HPLC lipidation profile of the lipidated proteins, wherein a first peak (P1+P2) represents the Lip of formula (I) with two lipids being C16:0 and one being C16:1, a second peak (P3) represents the Lip of formula (I) with two lipids being C16:0 and one being C17:1, a third peak (P4) represents the Lip of formula (I) with two lipids being C16:0 and one being C18:1 and a fourth peak (P5+P6) represents the Lip of formula (I) with two lipids being C16:0 and one being cycC19, wherein peaks P1+P2, P3, P4 and P5+P6 comprise 23±10%, 41±10%, 25±10% and 12±10% of the total lipidated proteins, respectively. Preferably, the peaks P1+P2, P3, P4 and P5+P6 comprise 23±5%, 41±5%, 25±5% and 12±5% of the total lipidated proteins, respectively.

In one embodiment, step a) comprising culturing E. coli cells producing a lipidated protein is separated into at least two phases: i) the batch phase and ii) the feed phase. The batch phase is defined as a phase of initial growth of E. coli following seeding of the large volume of medium in the fermenter e.g., from about 40 L to up to about 2000 L. The batch phase lasts for a period of several hours e.g., 8 to 24 hours, or up to about 12 hours. The feed phase is defined as the phase during which the recombinant protein is expressed as a result of induction, i.e., by the addition of and inducing agent, e.g. IPTG. The feed phase is typically shorter than the batch phase lasting for about e.g. between 3 and 8 hours, especially about 7 hours.

In accordance with the present invention, the lipidated protein or lipoprotein may be any naturally occurring or engineered protein of CD origin, such as a CD protein or fragment thereof, fusion protein or a heterodimer, which has covalently attached one or more lipids. Preferably, the N-terminal amino acid of the protein is a cysteine and the lipidated protein comprises three lipids. The term “lipidated protein” refers to a protein that is not lipidated in its native form, but is modified, e.g., by adding a lipoprotein signal peptide, so that it is produced in lipidated form. Lipoprotein signal peptides (or lipid signal peptides), found in natural lipoproteins, are known in the art. Lipidation of a protein with an N-terminal lipidation signal sequence, such as those present on a nascent CD polypeptide or the particular CD polypeptides herein described, occurs in the E. coli expression vector by the step-wise action of the enzymes diacylglyceryl transferase, signal peptidase II and transacylase, respectively. The first step is the transfer of a diacylglyceride to the cysteine sulfhydryl group of the unmodified pro-protein, followed by the cleavage of the signal peptide by signal peptidase II and, finally, the acylation of the [alpha]-amino group of the N-terminal cysteine of the protein. The result is the placement of one lipid and a glycerol group substituted with two further lipids on the N-terminal cysteine residue of the polypeptide. The lipidation signal sequence, which is cleaved off during lipidation, is not present in the final polypeptide sequence.

According to the present invention, the lipidated protein has one, two or three lipids attached to a glycerol and the amino group of the N-terminal cysteine. The lipid moieties, along with the glycerol group of the lipidated protein, is also referred to as “Lip”. Lip comprises one, two or three lipids, such as C₁₄₋₂₀ alkyl and/or C₁₄₋₂₀ alkenyl, attached to a glycerol and the N-terminal cysteine of the polypeptide of the invention, particularly wherein the lipidated protein has one lipid and a glycerol substituted with two lipids attached to the amino group of the N-terminal cysteine of the protein and/or particularly wherein the three acyl residues of the lipids are independently selected from C₁₄₋₂₀ alkyl and/or C₁₄₋₂₀ alkenyl. Preferably, Lip is a moiety of formula (I) below,

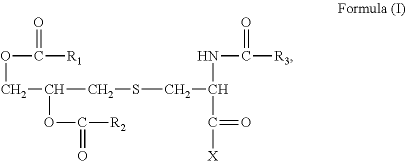

in which R₁, R₂ and/or R₃ are independently selected from C₁₄-C₂₀ alkyl or C₁₄-C₂₀ alkenyl and in which X is an amino acid sequence attached to the cysteine residue shown in Formula (I). More preferably, Lip plus the N-terminal cysteine of the polypeptide is N-palmitoyl-S-(2RS)-2,3-bis-(palmitoyloxy) propyl cysteine (referred to herein as “Pam3Cys”) and is connected via the carbonyl C of the N-terminal cysteine to said amino acid sequence of the invention. In Formula (I) above R1, R2 and R3 would be palmitoyl moieties (16:0) and X is an amino acid sequence attached to the cysteine residue.

The typical lipidation profile of the lipidated protein of the present invention is as follows: about 50% (e.g. 40-60%) of the fatty acids of the lipidation sites are palmitic acid (C16:0), about 10 to 20% are mono-unsaturated fatty acids comprising 17 C atoms (C17:1), about 10 to 20% are mono-unsaturated fatty acids comprising 18 C atoms (C18:1) (oleic acid), about 5 to 20% (e.g. about 8 to 15%) are mono-unsaturated fatty acids comprising 16 C atoms (C16:1) (palmitoleic acid) and about 0 to 10% are other fatty acids, such as e.g. about 1 to 5% are cyclopropane-comprising fatty acids having 19 C atoms (cycC19) (lactobacillic acid). Other fatty acids such as C14:0 and C15:0 are present at an even lower amount.

Detailed characterization of the lipidated protein can be done by liquid-chromatography (RP-HPLC) and mass spectrometry (LC-MS). Separation is performed on a Zorbax 300SB-CN narrow bore column (2.1×150 mm, 5 μm; Agilent) in a water/acetonitrile gradient (0.1% formic acid) from 20 to 80% acetonitrile within 15 minutes (flow rate 0.2 mL/min, column temperature 60° C.). The obtained mass spectra (Waters micromass ZQ, ESI-MS) are de-convoluted by MaxEnt software (Waters Corporation) as described in WO202105022 A1 incorporated herein by reference.

The production conditions (pressure and pH optionally in combination with trace elements and antifoam agent) may be optimized to obtain a suitable lipidation profile of the produced lipidated proteins.

Critical cultivation parameters which have an influence on the lipidation pattern are pH and headspace pressure applied during cultivation to facilitate oxygen supply.

Also trace elements added during cultivation have an impact on the lipidation patterns of recombinant proteins, with different concentrations resulting in different lipidation profiles. A trace element is a chemical element having a very low concentration or availability. The usual cations that qualify as trace elements in bacterial nutrition are Mn, Co, Zn, Cu, and Mo (Todar, K; Todar's Online Textbook of Bacteriology; Nutrition and Growth of Bacteria, p.1, http://textbookofbacteriology.net/nutgro.html; accessed 11 Mar. 2021). Iron (Fe) is present in higher amounts in bacteria and, while it is not considered a trace element per se, the environmental availability of Fe profoundly influences bacterial processes, such as, e.g., the expression of iron-requiring bacterial proteins (Andrews, SC et al. Bacterial iron homeostasis (2003) FEMS Microbiology Reviews 27:215-237). As such, for the purposes of the invention, Fe is considered as a trace element, i.e., is included in a trace element solution to supplement nutrition of bacteria during fermentation.

In a preferred embodiment of the present invention, a trace element (TE) solution (also referred to herein as trace element (TE) cocktail) is added during culturing step a), particularly during batch phase i) and/or feed phase ii). Trace elements, also called micronutrients, encompass any chemical element required by living organisms that is less than 0.1 percent by volume and are usually as part of a vital enzyme (a cell-produced catalytic protein). Preferably, the trace element (TE) solution comprises Fe, Co, Cu, Zn and/or Mo ions. In a preferred embodiment, the TE solution comprises the trace elements in the form of Iron(III)chloride hexahydrate, cobalt(II)chloride hexahydrate, copper(II)chloride dehydrate, zinc chloride and sodium molybdate dehydrate. In one embodiment, the TE solution further comprises boric acid and/or hydrochloric acid (HCl). Alternative salts of Fe, Co, Cu, Zn and Mo may be suitable as well. In a more preferred embodiment, the TE stock solution comprises 1.6 g/L Iron(III)chloride hexahydrate, 0.27 g/L cobalt(II)chloride hexahydrate, 0.127 g/L copper(II)chloride dehydrate, 0.2 g/L zinc chloride, 0.2 g/L sodium molybdate dihydrate, 0.05 g/L boric acid and 16.7 mL/L hydrochloric acid. The TE stock solution may be added to the medium at a dilution of 1/10000 to 1/10 (mL TE. mL culture medium), such as 1/1000. It has been found that higher amounts of trace elements may be needed in the feed phase rather than the batch phase. Accordingly, suitable dilutions in the feed phase, i.e., added to the feed phase medium, may be from 1/10 to 1/60, such as 1/12, 1/24, 1/36, 1/48 and 1/60. Particularly preferred in the feed phase are higher amounts of TE solution, i.e., dilutions of around 1/12, 1/24, 1/36 or 1/48. Suitable dilutions in the batch phase; i.e., added to the batch phase medium, may be from 1/10000 to 1/1600, such as 1/8000, 1/6400, 1/3200 and 1/1600, preferably around 1/8000, such as from 1/7500 to 1/8500. In general, the volume of the feed phase medium is approximately 15% of the volume of the batch medium. For example, in a lab scale fermentation run, the batch phase volume may be about 8 L and the feed phase about 1.2 L.

In a further preferred embodiment of the present invention, an anti-foam agent is present during culturing step a). An anti-foaming agent is a chemical additive that prevents the formation of foam in industrial process liquids. Foam occurs in bioprocesses due to the introduction of gases into the culture medium and is further stabilized by proteins produced by organisms in the culture. In formats of larger scale, foaming is a problem that is particularly acute due to gassing used to maintain appropriate dissolved oxygen (DO) concentrations. Foaming can lead to reduced process productivity since bursting bubbles can damage proteins, result in loss of sterility if the foam escapes the bioreactor or lead to over-pressure if a foam-out blocks an exit filter. To prevent the formation of foam, one or more anti-foam agents may be employed in the method of the present invention. Anti-foam agents can be classified as either hydrophobic solids dispersed in carrier oil, aqueous suspensions/emulsions, liquid single components or solids and may contain surfactants. Examples for suitable anti-foam agents include without limitation silicone oil (S184), polypropylene glycol (PPG), such as PPG-2000, silicone oil/PPG mixture, and an emulsion containing 10% S184. A particular preferred anti-foam agent is PPG-2000.

In one embodiment, the anti-foam agent may be present during both i) batch phase and ii) feed phase. The anti-foam agent is especially suitable during the exponential phase of E. coli growth (feed phase) of the culturing. Therefore, the anti-foam agent is preferably added and/or increased in concentration during the exponential phase of E. coli growth (feed phase). It has been found that repeated or continuous addition of the anti-foam agent is particularly useful in the production of the lipidated proteins with the method of the present invention. Accordingly, the anti-foam agent is added repeatedly during culturing, especially in a bolus twice during the feed phase, preferably once before induction and once after induction. Alternatively, the anti-foam agent is added continuously during the feed phase (exponentially). In one embodiment, the anti-foam agent is present in both the batch phase and feed phase media. Optimization of the amount of AF and the time and mode of administration can improve batch-to-batch consistency, which is crucial for bioprocess production scale.

Accordingly, the cultivation parameters pH and headspace pressure, optionally in combination with trace elements and/or an anti-foam agent, may be used to modulate the lipid peak pattern of recombinantly expressed lipidated protein in order to obtain the indicated lipidation pattern, in which about 40-60% of the fatty acids are palmitic acid (16:0), about 10 to 20% are mono-unsaturated fatty acids comprising 17 C atoms, about 10 to 20% are mono-unsaturated fatty acids comprising 18 C atoms, about 5 to 20% are mono-unsaturated fatty acids comprising 16 C atoms and about 0 to 10% are other fatty acids, particularly in which about 50% of the fatty acids are palmitic acid, about 10 to 20% are mono-unsaturated fatty acids comprising 17 C atoms, about 10 to 20% are mono-unsaturated fatty acids comprising 18 C atoms, about 8 to 15% are mono-unsaturated fatty acids comprising 16 C atoms and about 1 to 5% are cyclopropane-comprising fatty acids having 19 C atoms, or
in which 23±10% of the total lipidated proteins comprise two palmitic acids (16:0) and one C16:1 fatty acid, 41±10% of the total lipidated proteins comprise two palmitic acids (16:0) and one and one C17:1 fatty acid, 25±10% of the total lipidated proteins comprise two palmitic acids (16:0) and one C18:1 fatty acid and 12±10% of the total lipidated proteins comprise two palmitic acids (16:0) and one cycC19 fatty acid; or
in which 18±10% of the total lipidated proteins comprise two palmitic acids (16:0) and one C16:1 fatty acid, 46±10% of the total lipidated proteins comprise two palmitic acids (16:0) and one and one C17:1 fatty acid, 20±10% of the total lipidated proteins comprise two palmitic acids (16:0) and one C18:1 fatty acid and 16±10% of the total lipidated proteins comprise two palmitic acids (16:0) and one cycC19 fatty acid.

According to one embodiment of the present invention, the lipidated protein is expressed in a host cell, namely an E. coli cell suitable for producing the protein in lipidated form, via conventional recombinant technology. Briefly, a DNA fragment encoding the protein is provided. The DNA fragment may be inserted into an E. coli expression vector to produce an expression plasmid. The expression plasmid may be introduced into a selected E. coli strain by transformation with the plasmid encompassing a nucleic acid sequence coding for the protein of interest (e.g. vector pET28b(+)) to allow for the production of the lipidated protein. Transformation may be done by heat shock. Positive transformants are cultured under suitable conditions for protein expression. Suitable cells may be E. coli BL21(DE3), Genotype F⁻ompT hsdSB(rB⁻mB⁻) gal dcm (DE3) (Invitrogen). The lipidated protein thus expressed can be isolated from the E. coli cells and its lipidation status may be confirmed via methods known in the art, e.g., immunoblotting with an anti-lipoprotein antibody or mass spectrometry.

Cells may be cultured for a time and under conditions allowing for the production of the lipidated protein. The minimal volume in which the cells are cultured is 40 L. In a further preferred embodiment, the volume is at least 100 L, at least 200 L or at least 300 L. The maximal volume may be 2000 L or 1000 L.

As detailed above, the pH and pressure will be selected to obtain the intended lipidation profile. In a preferred embodiment, the amount of trace elements and/or anti-foam agents are also defined during culturing. Throughout the cultivation the dissolved oxygen level (DO) will usually be maintained at a constant level. The process may be monitored by in-process controls for several fermenter parameters like temperature, pH, DO, aeration rate, agitation rate, feeding rate, acid/base consumption and headspace pressure.

The produced lipidated protein is harvested by extraction from E. coli cell culture, with e.g. a detergent, such as Triton X-114. For this, cells may be broken, e.g. by resuspending in lysis buffer and/or disrupting by high pressure homogenization (e.g. two passages at 800 bar). The lipid moiety of the protein may be utilized to selectively extract the proteins with detergent, such as Triton X-114. During solubilization the nonionic detergent replaces most lipid molecules in contact with the hydrophobic domain or lipid moiety and leads to the formation of a soluble protein-detergent mixed micelle. As the temperature is raised, the micellar molecular weight increases, and the solution turns suddenly turbid (cloud point). At this temperature a microscopic phase separation of the solution caused by formation of larger micelle aggregates occurs. These larger micelle aggregates become immiscible with water and start to separate from the water phase. This phase separation occurs until two clear phases are formed. Hydrophilic proteins are recovered in the aqueous phase, whereas hydrophobic proteins are enriched in the detergent phase after separation. The obtained proteins may be further purified as known in the art (e.g. extraction, chromatographic methods, ultrafiltration, etc.). Finally, the purified protein may be stored in a suitable solution (e.g. isotonic saline comprising excipients or stabilizers, pH 6.2 to 7.2) until use.

The lipidated protein or polypeptide of the present invention is an immunogenic protein or polypeptide derived from the pathogenic bacterium Clostridium difficile, particularly the protein comprising an immunogenic polypeptide fragment of Clostridium difficile toxin B, or an immunogenic polypeptide fragment of Clostridium difficile toxin A, or both toxin A and toxin B sequences. Preferably, the lipidated immunogenic protein or polypeptide is a sole active agent of the immunogenic composition of the present invention. Alternatively, the lipidated immunogenic protein or polypeptide is used in combination with one or more further antigen(s), especially Clostridium difficile antigen(s).

Subsequently, the present invention also includes compositions (or vaccines) and formulations comprising at least one lipidated Clostridium difficile toxin A or toxin B protein or polypeptide.

Clostridium difficile is the leading cause of nosocomial antibiotic associated diarrhea and has become a major health problem in hospitals, nursing home and other care facilities. C. difficile associated disease (CDAD) is induced by the disruption of the normal colonic flora, usually the result of the administration of antibiotics. Following exposure to C. difficile spores in the environment, the organism may colonize the intestinal mucosa where the production of disease causing toxins can result in CDAD. Disease may range from mild uncomplicated diarrhea to severe pseudomembranous colitis and toxic megacolon. CDAD is the result of the actions of two exotoxins produced by C. difficile, toxin A and toxin B (also referred to as CTA and CTB, respectively). Both toxins are high molecular weight (˜300 kDa) secreted proteins that possess multiple functional domains (Voth D E and Ballard J D, Clinical Microbiology Reviews 18:247-263 (2005)). The N-terminal domain of both toxins contains ADP-glucosyltransferase activity that modifies Rho-like GTPases. This modification causes a loss of actin polymerization and cytoskeletal changes resulting in the disruption of the colonic epithelial tight junctions. This leads to excessive fluid exudation into the colon and a resulting diarrhea. The central domain contains a hydrophobic domain and is predicted to be involved in membrane transport. The C-terminal domain of both toxins contain multiple homologous regions called repeating units (RUs) that are involved in toxin binding to target cells (Ho et al, (2005) PNAS 102(51):18373-18378). Throughout the present description, the term “C-terminal domain of toxin A or toxin B” is equivalent to the term “cell-binding domain of toxin A or toxin B”. The repeating units are classified as either short (21-30 amino acids) or long (˜50 amino acids). Repeating units combine to form clusters, each usually containing one long and 3-5 short repeating units. The full-length toxin A possesses 39 repeating units (ARUs) organized into 8 clusters (Dove et al. Infect. Immun. 58:480-488 (1990), while the full-length toxin B contains 24 repeating units (BRUs) organized into 5 clusters (Barroso et al., Nucleic Acids Res. 18:4004 (1990); Eichel-Streiber et al., Gene 96:107-113 (1992)). Further details on Clostridium difficile toxin proteins and toxin based vaccines may be found e.g. in WO2012028741A1 and EP2753352B2. In accordance with the present invention, the toxin A and toxin B proteins are preferably derived from the Clostridium difficile strain 630 (ATCC BAA-1382).

In one embodiment, the lipidated immunogenic protein or polypeptide of the present invention comprises the non-toxic Clostridium difficile toxin A full-length protein or immunogenic fragment thereof or Clostridium difficile toxin B full-length protein of fragment thereof. Especially, the non-toxic Clostridium difficile toxin A or toxin B full-length protein is in a mutated form or toxoid, as described e.g. in WO2012143902 (Pfizer), WO2021255690 (Pfizer) and WO2014144594 (Sanofi).

In one embodiment, the immunogenic composition (or vaccine) of the present invention comprises the lipidated Clostridium difficile toxin A full-length protein or a fragment thereof. The Clostridium difficile toxin A full-length protein has the sequence as set forth in SEQ ID NO: 1 shown below:

Toxin A full-length
SEQ ID NO: 1
MSLISKEELIKLAYSIRPRENEYKTILTNLDEYNKLTTNNNENKYLQLKK
LNESIDVFMNKYKTSSRNRALSNLKKDILKEVILIKNSNTSPVEKNLHFV
WIGGEVSDIALEYIKQWADINAEYNIKLWYDSEAFLVNTLKKAIVESSTT
EALQLLEEEIQNPQFDNMKFYKKRMEFIYDRQKRFINYYKSQINKPTVPT
IDDIIKSHLVSEYNRDETVLESYRTNSLRKINSNHGIDIRANSLFTEQEL
LNIYSQELLNRGNLAAASDIVRLLALKNFGGVYLDVDMLPGIHSDLFKTI
SRPSSIGLDRWEMIKLEAIMKYKKYINNYTSENFDKLDQQLKDNFKLIIE
SKSEKSEIFSKLENLNVSDLEIKIAFALGSVINQALISKQGSYLTNLVIE
QVKNRYQFLNQHLNPAIESDNNFTDTTKIFHDSLFNSATAENSMFLTKIA
PYLQVGFMPEARSTISLSGPGAYASAYYDFINLQENTIEKTLKASDLIEF
KFPENNLSQLTEQEINSLWSFDQASAKYQFEKYVRDYTGGSLSEDNGVDF
NKNTALDKNYLLNNKIPSNNVEEAGSKNYVHYIIQLQGDDISYEATCNLF
SKNPKNSIIIQRNMNESAKSYFLSDDGESILELNKYRIPERLKNKEKVKV
TFIGHGKDEFNTSEFARLSVDSLSNEISSFLDTIKLDISPKNVEVNLLGC
NMFSYDFNVEETYPGKLLLSIMDKITSTLPDVNKNSITIGANQYEVRINS
EGRKELLAHSGKWINKEEAIMSDLSSKEYIFFDSIDNKLKAKSKNIPGLA
SISEDIKTLLLDASVSPDTKFILNNLKLNIESSIGDYIYYEKLEPVKNII
HNSIDDLIDEFNLLENVSDELYELKKLNNLDEKYLISFEDISKNNSTYSV
RFINKSNGESVYVETEKEIFSKYSEHITKEISTIKNSIITDVNGNLLDNI
QLDHTSQVNTLNAAFFIQSLIDYSSNKDVLNDLSTSVKVQLYAQLFSTGL
NTIYDSIQLVNLISNAVNDTINVLPTITEGIPIVSTILDGINLGAAIKEL
LDEHDPLLKKELEAKVGVLAINMSLSIAATVASIVGIGAEVTIFLLPIAG
ISAGIPSLVNNELILHDKATSVVNYFNHLSESKKYGPLKTEDDKILVPID
DLVISEIDFNNNSIKLGTCNILAMEGGSGHTVTGNIDHFFSSPSISSHIP
SLSIYSAIGIETENLDFSKKIMMLPNAPSRVFWWETGAVPGLRSLENDGT
RLLDSIRDLYPGKFYWRFYAFFDYAITTLKPVYEDTNIKIKLDKDTRNFI
MPTITTNEIRNKLSYSFDGAGGTYSLLLSSYPISTNINLSKDDLWIFNID
NEVREISIENGTIKKGKLIKDVLSKIDINKNKLIIGNQTIDFSGDIDNKD
RYIFLTCELDDKISLIIEINLVAKSYSLLLSGDKNYLISNLSNIIEKINT
LGLDSKNIAYNYTDESNNKYFGAISKTSQKSIIHYKKDSKNILEFYNDST
LEFNSKDFIAEDINVFMKDDINTITGKYYVDNNTDKSIDFSISLVSKNQV
KVNGLYLNESVYSSYLDFVKNSDGHHNTSNFMNLFLDNISFWKLFGFENI
NFVIDKYFTLVGKTNLGYVEFICDNNKNIDIYFGEWKTSSSKSTIFSGNG
RNVVVEPIYNPDTGEDISTSLDFSYEPLYGIDRYINKVLIAPDLYTSLIN
INTNYYSNEYYPEIIVLNPNTFHKKVNINLDSSSFEYKWSTEGSDFILVR
YLEESNKKILQKIRIKGILSNTQSFNKMSIDFKDIKKLSLGYIMSNFKSF
NSENELDRDHLGFKIIDNKTYYYDEDSKLVKGLININNSLFYFDPIEFNL
VTGWQTINGKKYYFDINTGAALISYKIINGKHFYFNNDGVMQLGVFKGPD
GFEYFAPANTQNNNIEGQAIVYQSKFLTLNGKKYYFDNDSKAVTGWRIIN
NEKYYFNPNNAIAAVGLQVIDNNKYYFNPDTAIISKGWQTVNGSRYYFDT
DTAIAFNGYKTIDGKHFYFDSDCVVKIGVFSTSNGFEYFAPANTYNNNIE
GQAIVYQSKFLTLNGKKYYFDNNSKAVTGWQTIDSKKYYFNTNTAEAATG
WQTIDGKKYYFNTNTAEAATGWQTIDGKKYYFNTNTAIASTGYTIINGKH
FYFNTDGIMQIGVFKGPNGFEYFAPANTDANNIEGQAILYQNEFLTLNGK
KYYFGSDSKAVTGWRIINNKKYYFNPNNAIAAIHLCTINNDKYYFSYDGI
LQNGYITIERNNFYFDANNESKMVTGVFKGPNGFEYFAPANTHNNNIEGQ
AIVYQNKFLTLNGKKYYFDNDSKAVTGWQTIDGKKYYFNLNTAEAATGWQ
TIDGKKYYFNLNTAEAATGWQTIDGKKYYFNTNTFIASTGYTSINGKHFY
FNTDGIMQIGVFKGPNGFEYFAPANTHNNNIEGQAILYQNKFLTLNGKKY
YFGSDSKAVTGLRTIDGKKYYFNTNTAVAVTGWQTINGKKYYFNTNTSIA
STGYTIISGKHFYFNTDGIMQIGVFKGPDGFEYFAPANTDANNIEGQAIR
YQNRFLYLHDNIYYFGNNSKAATGWVTIDGNRYYFEPNTAMGANGYKTID
NKNFYFRNGLPQIGVFKGSNGFEYFAPANTDANNIEGQAIRYQNRFLHLL
GKIYYFGNNSKAVTGWQTINGKVYYFMPDTAMAAAGGLFEIDGVIYFFGV
DGVKAPGIYG

In another embodiment, the immunogenic composition or vaccine of the present invention comprises the lipidated Clostridium difficile toxin B full-length protein or a fragment thereof. The Clostridium difficile toxin B full-length protein has the sequence as set forth in SEQ ID NO: 2 shown below:

Toxin B full-length
SEQ ID NO: 2
MSLVNRKQLEKMANVRFRTQEDEYVAILDALEEYHNMSENTVVEKYLKLK
DINSLTDIYIDTYKKSGRNKALKKFKEYLVTEVLELKNNNLTPVEKNLHF
VWIGGQINDTAINYINQWKDVNSDYNVNVFYDSNAFLINTLKKTVVESAI
NDTLESFRENLNDPRFDYNKFFRKRMEIIYDKQKNFINYYKAQREENPEL
IIDDIVKTYLSNEYSKEIDELNTYIEESLNKITQNSGNDVRNFEEFKNGE
SFNLYEQELVERWNLAAASDILRISALKEIGGMYLDVDMLPGIQPDLFES
IEKPSSVTVDFWEMTKLEAIMKYKEYIPEYTSEHFDMLDEEVQSSFESVL
ASKSDKSEIFSSLGDMEASPLEVKIAFNSKGIINQGLISVKDSYCSNLIV
KQIENRYKILNNSLNPAISEDNDFNTTTNTFIDSIMAEANADNGRFMMEL
GKYLRVGFFPDVKTTINLSGPEAYAAAYQDLLMFKEGSMNIHLIEAMSLV
NRKQLEKMANVRFRTQEDEYVAILDALEEYHNMSENTVVEKYLKLKDINS
LTDIYIDTYKKSGRNKALKKFKEYLVTEVLELKNNNLTPVEKNLHFVWIG
GQINDTAINYINQWKDVNSDYNVNVFYDSNAFLINTLKKTVVESAINDTL
ESFRENLNDPRFDYNKFFRKRMEIIYDKQKNFINYYKAQREENPELIIDD
IVKTYLSNEYSKEIDELNTYIEESLNKITQNSGNDVRNFEEFKNGESFNL
YEQELVERWNLAAASDILRISALKEIGGMYLDVDMLPGIQPDLFESIEKP
SSVTVDFWEMTKLEAIMKYKEYIPEYTSEHFDMLDEEVQSSFESVLASKS
DKSEIFSSLGDMEASPLEVKIAFNSKGIINQGLISVKDSYCSNLIVKQIE
NRYKILNNSLNPAISEDNDFNTTTNTFIDSIMAEANADNGRFMMELGKYL
RVGFFPDVKTTINLSGPEAYAAAYQDLLMFKEGSMNIHLIEADLRNFEIS
KTNISQSTEQEMASLWSFDDARAKAQFEEYKRNYFEGSLGEDDNLDFSQN
IVVDKEYLLEKISSLARSSERGYIHYIVQLQGDKISYEAACNLFAKTPYD
SVLFQKNIEDSEIAYYYNPGDGEIQEIDKYKIPSIISDRPKIKLTFIGHG
KDEFNTDIFAGFDVDSLSTEIEAAIDLAKEDISPKSIEINLLGCNMFSYS
INVEETYPGKLLLKVKDKISELMPSISQDSIIVSANQYEVRINSEGRREL
LDHSGEWINKEESIIKDISSKEYISFNPKENKITVKSKNLPELSTLLQEI
RNNSNSSDIELEEKVMLTECEINVISNIDTQIVEERIEEAKNLTSDSINY
IKDEFKLIESISDALCDLKQQNELEDSHFISFEDISETDEGFSIRFINKE
TGESIFVETEKTIFSEYANHITEEISKIKGTIFDTVNGKLVKKVNLDTTH
EVNTLNAAFFIQSLIEYNSSKESLSNLSVAMKVQVYAQLFSTGLNTITDA
AKVVELVSTALDETIDLLPTLSEGLPIIATIIDGVSLGAAIKELSETSDP
LLRQEIEAKIGIMAVNLTTATTAIITSSLGIASGFSILLVPLAGISAGIP
SLVNNELVLRDKATKVVDYFKHVSLVETEGVFTLLDDKIMMPQDDLVISE
IDFNNNSIVLGKCEIWRMEGGSGHTVTDDIDHFFSAPSITYREPHLSIYD
VLEVQKEELDLSKDLMVLPNAPNRVFAWETGWTPGLRSLENDGTKLLDRI
RDNYEGEFYWRYFAFIADALITTLKPRYEDTNIRINLDSNTRSFIVPIIT
TEYIREKLSYSFYGSGGTYALSLSQYNMGINIELSESDVWIIDVDNVVRD
VTIESDKIKKGDLIEGILSTLSIEENKIILNSHEINFSGEVNGSNGFVSL
TFSILEGINAIIEVDLLSKSYKLLISGELKILMLNSNHIQQKIDYIGFNS
ELQKNIPYSFVDSEGKENGFINGSTKEGLFVSELPDVVLISKVYMDDSKP
SFGYYSNNLKDVKVITKDNVNILTGYYLKDDIKISLSLTLQDEKTIKLNS
VHLDESGVAEILKFMNRKGNTNTSDSLMSFLESMNIKSIFVNFLQSNIKF
ILDANFIISGTTSIGQFEFICDENDNIQPYFIKFNTLETNYTLYVGNRQN
MIVEPNYDLDDSGDISSTVINFSQKYLYGIDSCVNKVVISPNIYTDEINI
TPVYETNNTYPEVIVLDANYINEKINVNINDLSIRYVWSNDGNDFILMST
SEENKVSQVKIRFVNVFKDKTLANKLSFNFSDKQDVPVSEIILSFTPSYY
EDGLIGYDLGLVSLYNEKFYINNFGMMVSGLIYINDSLYYFKPPVNNLIT
GFVTVGDDKYYFNPINGGAASIGETIIDDKNYYFNQSGVLQTGVFSTEDG
FKYFAPANTLDENLEGEAIDFTGKLIIDENIYYFDDNYRGAVEWKELDGE
MHYFSPETGKAFKGLNQIGDYKYYFNSDGVMQKGFVSINDNKHYFDDSGV
MKVGYTEIDGKHFYFAENGEMQIGVFNTEDGFKYFAHHNEDLGNEEGEEI
SYSGILNFNNKIYYFDDSFTAVVGWKDLEDGSKYYFDEDTAEAYIGLSLI
NDGQYYFNDDGIMQVGFVTINDKVFYFSDSGIIESGVQNIDDNYFYIDDN
GIVQIGVFDTSDGYKYFAPANTVNDNIYGQAVEYSGLVRVGEDVYYFGET
YTIETGWIYDMENESDKYYFNPETKKACKGINLIDDIKYYFDEKGIMRTG
LISFENNNYYFNENGEMQFGYINIEDKMFYFGEDGVMQIGVFNTPDGFKY
FAHQNTLDENFEGESINYTGWLDLDEKRYYFTDEYIAATGSVIIDGEEYY
FDPDTAQLVISE

In yet another embodiment, the lipidated immunogenic protein or polypeptide of the present invention comprises or consists of the C-terminal domain (cell-binding domain) of toxin A or the C-terminal domain (cell-binding domain) of toxin B, or fragments thereof. According to the present invention, the cell-binding domain of toxin A or toxin B corresponds to the C-terminal protein sequence comprising repeating units, ARU and BRU, respectively, also named the C-terminal repeat domain. Throughout the present disclosure, all these terms are used interchangeably. The C-terminal domain of toxin A has the amino acid sequence of SEQ ID NO: 3 and the C-terminal domain of toxin B has the amino acid sequence of SEQ ID NO: 4 shown below:

Toxin A C-terminal domain
SEQ ID NO: 3
GLININNSLFYFDPIEFNLVTGWQTINGKKYYFDINTGAALISYKIINGK
HFYFNNDGVMQLGVFKGPDGFEYFAPANTQNNNIEGQAIVYQSKFLTLNG
KKYYFDNDSKAVTGWRIINNEKYYFNPNNAIAAVGLQVIDNNKYYFNPDT
AIISKGWQTVNGSRYYFDTDTAIAFNGYKTIDGKHFYFDSDCVVKIGVES
TSNGFEYFAPANTYNNNIEGQAIVYQSKFLTLNGKKYYFDNNSKAVTGWQ
TIDSKKYYFNTNTAEAATGWQTIDGKKYYFNTNTAEAATGWQTIDGKKYY
FNTNTAIASTGYTIINGKHFYFNTDGIMQIGVFKGPNGFEYFAPANTDAN
NIEGQAILYQNEFLTLNGKKYYFGSDSKAVTGWRIINNKKYYFNPNNAIA
AIHLCTINNDKYYFSYDGILQNGYITIERNNFYFDANNESKMVTGVFKGP
NGFEYFAPANTHNNNIEGQAIVYQNKFLTLNGKKYYFDNDSKAVTGWQTI
DGKKYYFNLNTAEAATGWQTIDGKKYYFNLNTAEAATGWQTIDGKKYYFN
TNTFIASTGYTSINGKHFYFNTDGIMQIGVFKGPNGFEYFAPANTHNNNI
EGQAILYQNKFLTLNGKKYYFGSDSKAVTGLRTIDGKKYYFNTNTAVAVT
GWQTINGKKYYFNTNTSIASTGYTIISGKHFYFNTDGIMQIGVFKGPDGF
EYFAPANTDANNIEGQAIRYQNRFLYLHDNIYYFGNNSKAATGWVTIDGN
RYYFEPNTAMGANGYKTIDNKNFYFRNGLPQIGVFKGSNGFEYFAPANTD
ANNIEGQAIRYQNRFLHLLGKIYYFGNNSKAVTGWQTINGKVYYFMPDTA
MAAAGGLFEIDGVIYFFGVDGVKAPGIYG
Toxin B C-terminal domain
SEQ ID NO: 4
GLIYINDSLYYFKPPVNNLITGFVTVGDDKYYFNPINGGAASIGETIIDD
KNYYFNQSGVLQTGVFSTEDGFKYFAPANTLDENLEGEAIDFTGKLIIDE
NIYYFDDNYRGAVEWKELDGEMHYFSPETGKAFKGLNQIGDYKYYFNSDG
VMQKGFVSINDNKHYFDDSGVMKVGYTEIDGKHFYFAENGEMQIGVFNTE
DGFKYFAHHNEDLGNEEGEEISYSGILNFNNKIYYFDDSFTAVVGWKDLE
DGSKYYFDEDTAEAYIGLSLINDGQYYFNDDGIMQVGFVTINDKVFYFSD
SGIIESGVQNIDDNYFYIDDNGIVQIGVFDTSDGYKYFAPANTVNDNIYG
QAVEYSGLVRVGEDVYYFGETYTIETGWIYDMENESDKYYFNPETKKACK
GINLIDDIKYYFDEKGIMRTGLISFENNNYYFNENGEMQFGYINIEDKMF
YFGEDGVMQIGVFNTPDGFKYFAHQNTLDENFEGESINYTGWLDLDEKRY
YFTDEYIAATGSVIIDGEEYYFDPDTAQLVISE

In one embodiment, the immunogenic composition of the present invention comprises the lipidated C. difficile toxin a polypeptide comprising a C. difficile toxin A cell-binding or C-terminal repeat domain (SEQ ID NO: 3) or fragment thereof, preferably that this lipidated C. difficile toxin A polypeptide lacks C. difficile toxin B cell-binding domain or C-terminal repeat domain sequence.

In yet another embodiment, the immunogenic composition of the present invention comprises the lipidated C. difficile toxin a polypeptide comprising a C. difficile toxin B cell-binding or C-terminal repeat domain (SEQ ID NO: 4) or fragment thereof, preferably that this lipidated C. difficile toxin B polypeptide lacks C. difficile toxin A cell-binding domain or C-terminal repeat domain sequence.

In another embodiment, the immunogenic composition or vaccine of the present invention comprises the lipidated polypeptide comprising or consisting of a sequence of SEQ ID NO: 5 derived from the C-terminal domain of Clostridium difficile toxin A shown below:

Lip-ToxA-His
SEQ ID NO: 5
LipCSSFVTGVFKGPNGFEYFAPANTHNNNIEGQAIVYQNKFLTLNGKKY
YFDNDSKAVTGWQTIDGKKYYFNLNTAEAATGWQTIDGKKYYFNLNTAEA
ATGWQTIDGKKYYFNTNTFIASTGYTSINGKHFYFNTDGIMQIGVFKGPN
GFEYFAPANTDANNIEGQAILYQNKFLTLNGKKYYFGSDSKAVTGLRTID
GKKYYFNTNTAVAVTGWQTINGKKYYFNTNTSIASTGYTIISGKHFYFNT
DGIMQIGVFKGPDGFEYFAPANTDANNIEGQAIRYQNRFLYLHDNIYYFG
NNSKAATGWVTIDGNRYYFEPNTAMGANGYKTIDNKNFYFRNGLPQIGVF
KGSNGFEYFAPANTDANNIEGQAIRYQNRFLHLLGKIYYFGNNSKAVTGW
QTINGKVYYFMPDTAMAAAGGLFEIDGVIYFFGVDGVKAPGIYGLEHHHH
HH

In yet another embodiment, the immunogenic composition or vaccine of the present invention comprises the lipidated polypeptide comprising or consisting of a sequence of SEQ ID NO: 6 derived from the C-terminal domain of Clostridium difficile toxin B shown below:

Lip-ToxB-His
SEQ ID NO: 6
LipCSSFNLITGFVTVGDDKYYFNPINGGAASIGETIIDDKNYYFNQSGV
LQTGVFSTEDGFKYFAPANTLDENLEGEAIDFTGKLIIDENIYYFDDNYR
GAVEWKELDGEMHYFSPETGKAFKGLNQIGDYKYYFNSDGVMQKGFVSIN
DNKHYFDDSGVMKVGYTEIDGKHFYFAENGEMQIGVFNTEDGFKYFAHHN
EDLGNEEGEEISYSGILNFNNKIYYFDDSFTAVVGWKDLEDGSKYYFDED
TAEAYIGLSLINDGQYYFNDDGIMQVGFVTINDKVFYFSDSGIIESGVQN
IDDNYFYIDDNGIVQIGVFDTSDGYKYFAPANTVNDNIYGQAVEYSGLVR
VGEDVYYFGETYTIETGWIYDMENESDKYYFNPETKKACKGINLIDDIKY
YFDEKGIMRTGLISFENNNYYFNENGEMQFGYINIEDKMFYFGEDGVMQI
GVFNTPDGFKYFAHQNTLDENFEGESINYTGWLDLDEKRYYFTDEYIAAT
GSVIIDGEEYYFDPDTAQLVISELEHHHHHH

In yet another embodiment, the immunogenic lipidated protein of the present invention is a Clostridium difficile toxin fusion protein. The Clostridium difficile toxin fusion protein comprises or consists of immunogenic fragments (parts) of toxin A and toxin B, preferably the full-length or a part of the C-terminal domain of toxin A fused to the full-length or a part of the C-terminal domain of toxin B.

Particularly, the Clostridium difficile toxin fusion protein is the lipidated form of C-TAB.G5 or C-TAB.G5.1 protein. The C-TAB.G5.1 comprises 19 repeating units of the C-terminal domain of toxin A (ARU) fused to 23 repeating units of the C-terminal domain of toxin B (BRU). Further information on the proteins C-TAB.G5 and C-TAB.G5.1 and their production is derivable from WO 2012028741 A1 and EP2753352 B2, wherein C-TAB.G5 and C-TAB.G5.1 correspond to SEQ ID NOs: 2 and 4, respectively. According to the present invention, the lipidated C-TAB.G5.1 protein (Lip-C-TAB.G5.1) has a sequence as set forth in SEQ ID NO: 7 shown below:

Lip-C-TAB.G5.1
SEQ ID NO: 7
LipCSSFVTGVFKGPNGFEYFAPANTHNNNIEGQAIVYQNKFLTLNGKKY
YFDNDSKAVTGWQTIDGKKYYFNLNTAEAATGWQTIDGKKYYFNLNTAEA
ATGWQTIDGKKYYFNTNTFIASTGYTSINGKHFYFNTDGIMQIGVFKGPN
GFEYFAPANTDANNIEGQAILYQNKFLTLNGKKYYFGSDSKAVTGLRTID
GKKYYFNTNTAVAVTGWQTINGKKYYFNTNTSIASTGYTIISGKHFYFNT
DGIMQIGVFKGPDGFEYFAPANTDANNIEGQAIRYQNRFLYLHDNIYYFG
NNSKAATGWVTIDGNRYYFEPNTAMGANGYKTIDNKNFYFRNGLPQIGVF
KGSNGFEYFAPANTDANNIEGQAIRYQNRFLHLLGKIYYFGNNSKAVTGW
QTINGKVYYFMPDTAMAAAGGLFEIDGVIYFFGVDGVKAPGIYGRSMHNL
ITGFVTVGDDKYYFNPINGGAASIGETIIDDKNYYFNQSGVLQTGVFSTE
DGFKYFAPANTLDENLEGEAIDFTGKLIIDENIYYFDDNYRGAVEWKELD
GEMHYFSPETGKAFKGLNQIGDYKYYFNSDGVMQKGFVSINDNKHYFDDS
GVMKVGYTEIDGKHFYFAENGEMQIGVFNTEDGFKYFAHHNEDLGNEEGE
EISYSGILNFNNKIYYFDDSFTAVVGWKDLEDGSKYYFDEDTAEAYIGLS
LINDGQYYFNDDGIMQVGFVTINDKVFYFSDSGIIESGVQNIDDNYFYID
DNGIVQIGVFDTSDGYKYFAPANTVNDNIYGQAVEYSGLVRVGEDVYYFG
ETYTIETGWIYDMENESDKYYFNPETKKACKGINLIDDIKYYFDEKGIMR
TGLISFENNNYYFNENGEMQFGYINIEDKMFYFGEDGVMQIGVFNTPDGF
KYFAHQNTLDENFEGESINYTGWLDLDEKRYYFTDEYIAATGSVIIDGEE
YYFDPDTAQLVISE

Alternatively, the lipidated Clostridium difficile toxin protein of the present invention may be a lipidated form of the Toxin A-Toxin B fusion protein described in WO2012163810 (GSK) or WO2018/170238 (Novavax) incorporated herein by reference.

The present invention also includes immunogenic variants of the lipidated C. difficile proteins (or polypeptides) described herein, especially the protein of any SEQ ID Nos. 1 to 7, wherein the protein variants have a sequence identity to SEQ ID Nos. 1 to 7 of at least about 80%, 81%, 82%, 83%, 84%, 85%, 86%, 87%, 88%, 89%, 90%, 91%, 92%, 93%, 94%, 95%, 96%, 97%, 98% or 99%. The immunogenic variant of the lipidated C. difficile protein or polypeptide described herein may be included into the immunogenic composition.

In one embodiment, the immunogenic composition of the present invention comprises an immunogenic lipidated protein or polypeptide with a sequence identity of at least about 80%, 81%, 82%, 83%, 84%, 85%, 86%, 87%, 88%, 89%, 90%, 91%, 92%, 93%, 94%, 95%, 96%, 97%, 98% or 99% to the C. difficile toxin A cell-binding domain of SEQ ID NO: 3.

In another embodiment, the immunogenic composition of the present invention comprises an immunogenic lipidated protein or polypeptide with a sequence identity of at least about 80%, 81%, 82%, 83%, 84%, 85%, 86%, 87%, 88%, 89%, 90%, 91%, 92%, 93%, 94%, 95%, 96%, 97%, 98% or 99% to the C. difficile toxin B cell-binding domain of SEQ ID NO: 4.

In another embodiment, the immunogenic composition of the present invention comprises an immunogenic lipidated protein or polypeptide with a sequence identity of at least about 80%, 81%, 82%, 83%, 84%, 85%, 86%, 87%, 88%, 89%, 90%, 91%, 92%, 93%, 94%, 95%, 96%, 97%, 98% or 99% to SEQ ID NO: 5.

In another embodiment, the immunogenic composition of the present invention comprises an immunogenic lipidated protein or polypeptide with a sequence identity of at least about 80%, 81%, 82%, 83%, 84%, 85%, 86%, 87%, 88%, 89%, 90%, 91%, 92%, 93%, 94%, 95%, 96%, 97%, 98% or 99% to SEQ ID NO: 6.

In another embodiment, the immunogenic composition of the present invention comprises an immunogenic lipidated protein or polypeptide with a sequence identity of at least 80%, at least about 80%, 81%, 82%, 83%, 84%, 85%, 86%, 87%, 88%, 89%, 90%, 91%, 92%, 93%, 94%, 95%, 96%, 97%, 98% or 99% to SEQ ID NO: 7.

In one preferred embodiment of the present invention, the lipidated C. difficile toxin A protein or polypeptide comprises or consists of a lipidated form of SEQ ID NO: 5 or a lipidated form of the full-length C. difficile toxin A C-terminal repeat domain sequence (SEQ ID NO: 3) or an immunogenic variant thereof with a sequence identity to SEQ ID Nos 3 or 5 of at least 80%, preferably at least 90%, more preferably at least 95%.

In yet another preferred embodiment of the present invention, the lipidated C. difficile toxin B protein or polypeptide comprises or consists of a lipidated form of SEQ ID NO: 6 or a lipidated form of the full-length C. difficile toxin B C-terminal repeat domain sequence (SEQ ID NO: 4) or an immunogenic variant thereof with a sequence identity to SEQ ID Nos 4 or 6 of at least 80%, preferably at least 90%, more preferably at least 95%.

In yet another preferred embodiment of the present invention, the lipidated protein is a lipidated Clostridium difficile toxin protein, particularly a lipidated form of a protein comprising the protein of SEQ ID NO: 7 (Lip-C-TAB.G5.1), or an immunogenic variant thereof with a sequence identity of at least about 80%, 85%, 90%, 95%, 96%, 97%, 98%, 99% to SEQ ID NO: 7.

Sequence identity is frequently measured in terms of percentage identity: the higher the percentage, the more identical the two sequences are. Homologs, orthologs, or variants of a polypeptide will possess a relatively high degree of sequence identity when aligned using standard methods. Methods of alignment of sequences for comparison are well known in the art. Various programs and alignment algorithms are described in: Smith & Waterman (Adv. Appl. Math. 2:482, 1981); Needleman & Wunsch (Mol. Biol. 48:443, 1970); Pearson & Lipman (Proc. Natl. Acad. Sci. USA 85:2444, 1988); Higgins & Sharp (Gene, 73:237-44, 1988); Higgins & Sharp (CABIOS 5: 151-3, 1989); Corpet et al. (Nuc. Acids Res. 16: 10881-90, 1988); Huang et al. (Computer Appls in the Biosciences 8: 155-65, 1992); Pearson et al. (Meth. Mol. Bio. 24:307-31, 1994) and Altschul et al. (J. Mol. Biol. 215:403-10, 1990), presents a detailed consideration of sequence alignment methods and homology calculations. Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. Preferably, the percentage sequence identity is determined over the full length of the sequence. For example, a peptide sequence that has 1166 matches when aligned with a test sequence having 1554 amino acids is 75.0 percent identical to the test sequence (1166÷1554* 100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

The NCBI Basic Local Alignment Search Tool (BLAST) (Altschul et al. 1990. Mol. Biol. 215:403) is available from several sources, including the National Center for Biotechnology Information (NCBI, Bethesda, MD) and on the internet, for use in connection with the sequence analysis programs BLASTP, BLASTN, BLASTX, TBLASTN and TBLASTX. A description of how to determine sequence identity using this program is available on the NCBI website on the internet. The BLAST and the BLAST 2.0 algorithm are also described in Altschul et al. (Nucleic Acids Res. 25: 3389-3402, 1977). Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff 1992. Proc. Natl. Acad. Sci. USA 89: 10915-10919).

Variants of a protein are typically characterized by possession of at least about 60%, for example at least about 80%, 85%, 90%, 91%, 92%, 93%, 94%, 95%, 96%, 97%, 98% or 99% sequence identity counted over at least defined number of amino acid residues of the reference sequence, over the full length of the reference sequence or over the full length alignment with the reference amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used.

One example of a useful algorithm is PILEUP. PILEUP uses a simplification of the progressive alignment method of Feng & Doolittle (Mol. Evol. 35: 351-360, 1987). The method used is similar to the method described by Higgins & Sharp (CABIOS 5: 151-153, 1989). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al. 1984. Nuc. Acids Res. 12: 387-395).

As used herein, reference to “at least 80% identity” refers to “at least 80%, at least 85%, at least 90%, at least 91%, at least 92%, at least 93%, at least 94%, at least 95%, at least 96%, at least 97%, at least 98%, at least 99%, or even 100% identity” to a specified reference sequence, e.g. to at least 50, 100, 150, 250, 500 amino acid residues of the reference sequence or to the full length of the sequence. As used herein, reference to “at least 90% identity” refers to “at least 90%, at least 91%, at least 92%, at least 93%, at least 94%, at least 95%, at least 96%, at least 97%, at least 98%, at least 99%, or even 100% identity” to a specified reference sequence, e.g. to at least 50, 100, 150, 250, 500 amino acid residues of the reference sequence or to the full length of the sequence.

The variant may have amino acid substitutions, deletions, or insertions as compared to the reference protein sequences SEQ ID Nos. 1 to 7. Additionally, the variant of the lipidated protein of the present invention usually have the same or similar level of immunogenicity as the original protein. An immunogenic variant can induce neutralizing antibodies recognizing the native protein of the pathogen.

Summarizing, the lipidated immunogenic protein or polypeptide of the present invention is a lipidated Clostridium difficile toxin protein, particularly a lipidated form of a protein comprising or consisting of the Clostridium difficile toxin A protein or polypeptide of SEQ ID NO: 1, 3 or 5 and/or the Clostridium difficile toxin B protein or polypeptide of SEQ ID NO: 2, 4 or 6 and/or the Clostridium difficile toxin fusion protein of SEQ ID NO: 7, or immunogenic variants thereof with a sequence identity of at least about 80%, 85%, 90%, 95%, 96%, 97%, 98%, 99% to SEQ ID Nos. 1 to 7, in which about 40-60% of the fatty acids are palmitic acid, about 10 to 20% are mono-unsaturated fatty acids comprising 17 C atoms, about 10 to 20% are mono-unsaturated fatty acids comprising 18 C atoms, about 5 to 20% are mono-unsaturated fatty acids comprising 16 C atoms and about 0 to 10% are other fatty acids, particularly in which about 50% of the fatty acids are palmitic acid, about 10 to 20% are mono-unsaturated fatty acids comprising 17 C atoms, about 10 to 20% are mono-unsaturated fatty acids comprising 18 C atoms, about 8 to 15% are mono-unsaturated fatty acids comprising 16 C atoms and about 1 to 5% are cyclopropane-comprising fatty acids having 19 C atoms; or in which 23±10% of the total lipidated proteins comprise two palmitic acids (16:0) and one C16:1 fatty acid, 41±10% of the total lipidated proteins comprise two palmitic acids (16:0) and one and one C17:1 fatty acid, 25±10% of the total lipidated proteins comprise two palmitic acids (16:0) and one C18:1 fatty acid and 12±10% of the total lipidated proteins comprise two palmitic acids (16:0) and one cycC19 fatty acid, or in which 18±10% of the total lipidated proteins comprise two palmitic acids (16:0) and one C16:1 fatty acid, 46±10% of the total lipidated proteins comprise two palmitic acids (16:0) and one and one C17:1 fatty acid, 20±10% of the total lipidated proteins comprise two palmitic acids (16:0) and one C18:1 fatty acid and 16±10% of the total lipidated proteins comprise two palmitic acids (16:0) and one cycC19 fatty acid.

According to the present invention, the lipidated protein is encompassed in an immunogenic composition. An immunogenic composition is any composition of material that elicits an immune response in a mammalian host when the immunogenic composition is injected or otherwise introduced. The immune response may be humoral, cellular, or both. A humoral response results in the production of specific antibodies by the mammalian host upon exposure to the immunogenic composition. A booster effect refers to an increased immune response to an immunogenic composition upon subsequent exposure of the mammalian host to the same immunogenic composition. The immunogenic compositions described herein are useful as vaccines able to provide a protective response in a human subject against an infection caused by the pathogenic bacterium Clostridium difficile.

The immunogenic composition may contain the isolated lipidated Clostridium difficile polypeptide or protein, an additional antigen, an adjuvant, and/or an excipient. Alternatively, the composition may consist essentially of the isolated lipidated Clostridium difficile polypeptide or protein without an adjuvant or other active ingredients but optionally comprises an excipient such as a carrier, buffer and/or stabilizer. According to the present invention, the immunogenic composition is pharmaceutically acceptable, which allows administration to a human.

In accordance with the described above, the present invention provides an immunogenic or pharmaceutical composition comprising the lipidated form of a protein comprising or consisting of the Clostridium difficile toxin B protein of SEQ ID NO: 2, 4 or 6, or immunogenic fragment thereof, or immunogenic variant thereof, and/or the lipidated form of a protein comprising or consisting of the Clostridium difficile toxin A protein of SEQ ID NO: 1, 3 or 5, or immunogenic fragment thereof, or immunogenic variant thereof, and/or the Clostridium difficile toxin fusion protein of SEQ ID NO: 7 (Lip-C-TAB.G5.1) or variant thereof, especially wherein an immunogenic variant thereof has a sequence identity to SEQ ID NOs: 1 to 7 of at least about 80%, 85%, 90%, 95%, 96%, 97%, 98%, or 99%.

In a preferred embodiment, the composition comprises the lipidated protein or polypeptide, which comprises the Clostridium difficile toxin B protein cell-binding domain or fragment thereof and lacks any Clostridium difficile toxin A cell-binding domain sequence.

In yet another embodiment, the composition comprises the lipidated protein or polypeptide, which comprises the Clostridium difficile toxin A protein cell-binding domain or fragment thereof and lacks any Clostridium difficile toxin B cell-binding domain sequence.

In a preferred embodiment, the composition comprises the lipidated Clostridium difficile toxin B polypeptide comprising or consisting of a lipidated form of SEQ ID NO: 6, or a lipidated from of the full-length Clostridium difficile toxin B C-terminal repeat domain of SEQ ID NO: 4, or an immunogenic variant thereof with a sequence identity to SEQ ID Nos. 4 or 6 of at least about 80%, preferably about 90%, more preferably about 95%.

In yet another preferred embodiment, the composition comprises the lipidated C. difficile toxin A polypeptide comprising or consisting of a lipidated form of SEQ ID NO: 5, or a lipidated form of the full length C. difficile toxin A C-terminal repeat domain sequence of SEQ ID NO: 3, or a variant thereof with a sequence identity to SEQ ID Nos 3 or 5 of at least about 80%, preferably about 90%, more preferably about 95%. In a preferred embodiment, the composition of the present invention comprises the lipidated C. difficile toxin B polypeptide comprising or consisting of a lipidated form of SEQ ID NO: 6.

In a more preferred embodiment, the composition of the present invention does not comprise a Clostridium difficile toxin A C-terminal repeat domain sequence.

In yet another embodiment of the present invention, the lipidated Clostridium difficile toxin B polypeptide or the lipidated Clostridium difficile toxin A polypeptide is the sole active agent in the immunogenic composition.

In another embodiment, the immunogenic composition may comprise a further Clostridium difficile antigen. For example, a further antigen may be selected from the group consisting of, but not limited to, Acd protein WP_009892971.1 (SEQ ID NO: 8), a C40 family peptidase WP_009890599.1 (SEQ ID NO: 9) as described in Goodarzi & Badmasti (2022)Microbial Pathogenesis 162,105372; Cwp66 and Cwp84 as described in Wright et al., (2008) J. Med. Microbiol. 57:750-756; FliC and/or FliD as described in Razim et al., Scientific Reports (2021) 11:9940; binary toxin CDTa and/or CDTb as described in Secore et al., PLOS, Jan. 26, (2017); BclA3 glycoprotein as described in Aubry et al., Vaccines 2020, 8, 73; PSII antigen as described in Lang et al., Infect & Immunity (2021) 89(11), and other lipoprotein-based Clostridium difficile vaccine candidates such as rlipoA-RBD as desribed in Huang et al., J Biomed. Sci. (2015) 22:65.

In a preferred embodiment, the immunogenic or pharmaceutical composition comprises the lipidated Clostridium difficile toxin protein or polypeptide of any SEQ ID Nos. 1 to 7, or an immunogenic variant thereof with a sequence identity of at least about 80%, 85%, 90%, 95%, 96%, 97%, 98%, 99% to any SEQ ID NOs: 1 to 7 produced by the method described herein the present invention.

In an additional embodiment, the composition of the present invention may optionally contain any pharmaceutically acceptable carrier or excipient, such as buffer substances, stabilizers or further active ingredients, especially ingredients known in connection with pharmaceutical compositions and/or vaccine production. The composition may comprise sodium phosphate, sodium chloride, L-methionine, sucrose and Polysorbate-20 (Tween 20) at a pH of 6.7+/−0.2. The pharmaceutically acceptable carriers and/or excipients useful in this invention are conventional and may include buffers, stabilizers, diluents, preservatives, and solubilizers. Remington's Pharmaceutical Sciences, by E. W. Martin, Mack Publishing Co., Easton, PA, 15th Edition (1975), describes compositions and formulations suitable for pharmaceutical delivery of the polypeptides herein disclosed. Acceptable carriers, excipients, or stabilizers are nontoxic to recipients at the dosages and concentrations that are administered. Carriers, excipients or stabilizers may further comprise buffers. Examples of excipients include, but are not limited to, carbohydrates (such as monosaccharide and disaccharide), sugars (such as sucrose, mannitol, and sorbitol), phosphate, citrate, antioxidants (such as ascorbic acid and methionine), preservatives (such as phenol, butanol, benzanol; alkyl parabens, catechol, octadecyldimethylbenzyl ammonium chloride, hexamethonium chloride, resorcinol, cyclohexanol, 3-pentanol, benzalkonium chloride, benzethonium chloride, and m-cresol), low molecular weight polypeptides, proteins (such as serum albumin or immunoglobulins), hydrophilic polymers amino acids, chelating agents (such as EDTA), salt-forming counter-ions, metal complexes (such as Zn-protein complexes), and non-ionic surfactants (such as TWEEN™ and polyethylene glycol).

The immunogenic or pharmaceutical composition of the present invention may further comprise an adjuvant. By “adjuvant” is meant any substance that is used to specifically or non-specifically potentiate an antigen-specific immune response, perhaps through activation of antigen presenting cells. An adjuvant may be administered with an antigen or may be administered by itself, either by the same route as that of the antigen or by a different route than that of the antigen. A single adjuvant molecule may have both adjuvant and antigen properties.

Examples of adjuvants include an oil emulsion (e.g., complete or incomplete Freund's adjuvant), Montanide incomplete Seppic adjuvant such as ISA, oil in water emulsion adjuvants such as the Ribi adjuvant system, syntax adjuvant formulation containing muramyl dipeptide, aluminum salt adjuvant (alum), polycationic polymer, especially polycationic peptide, especially polyarginine or a peptide containing at least two LysLeuLys motifs, especially KLKLLLLLKLK (SEQ ID NO: 13), immunostimulatory oligodeoxynucleotide (ODN) containing non-methylated cytosine-guanine dinucleotides (CpG) in a defined base context (e.g., as described in WO 96/02555) or ODNs based on inosine and cytidine (e.g., as described in WO 01/93903), or deoxynucleic acid containing deoxy-inosine and/or deoxyuridine residues (as described in WO 01/93905 and WO 02/095027), especially Oligo(dIdC)₁₃ (SEQ ID NO: 14, as described in WO 01/93903 and WO 01/93905), neuroactive compound, especially human growth hormone (described in WO 01/24822), or combinations thereof, a chemokine (e.g., defensins 1 or 2, RANTES, MIP1-α, MIP-2, interleukin-8, or a cytokine (e.g., interleukin-1β, -2, -6, -10 or -12; interferon-γ; tumor necrosis factor-α; or granulocyte-monocyte-colony stimulating factor) (reviewed in Nohria and Rubin, 1994), a muramyl dipeptide variant (e.g., murabutide, threonyl-MDP or muramyl tripeptide), synthetic variants of MDP, a heat shock protein or a variant, a variant of Leishmania major LeIF (Skeiky et al., 1995, J. Exp. Med. 181: 1527-1537), non-toxic variants of bacterial ADP-ribosylating exotoxins (bAREs) including variants at the trypsin cleavage site (Dickenson and Clements, (1995) Infection and Immunity 63 (5): 1617-1623) and/or affecting ADP-ribosylation (Douce et al., 1997) or chemically detoxified bAREs (toxoids), QS21, Quill A, N-acetylmuramyl-L-alanyl-D-isoglutamyl-L-alanine-2-[1,2-dipalmitoyl-s-glycero-3-(hydroxyphosphoryloxy)]ethylamide (MTP-PE) and compositions containing a metabolizable oil and an emulsifying agent.

In one particular embodiment, the adjuvant is an aluminium salt adjuvant (alum), preferably wherein said aluminium adjuvant is aluminium hydroxide. The aluminium hydroxide may comprise less than 1.25 ppb copper based on the weight of the composition. an adjuvant described in detail in WO2013/083726 or Schlegl et al. (Vaccine 33 (2015), pp. 5989-5996). Alum adjuvant promotes the induction of a predominantly T helper type 2 (Th2) immune response in an immunized subject.

In another particular embodiment, the adjuvant is CpG, preferably CpG 1018. As used herein, “CpG” refers to a cytosine-phospho-guanosine (CpG) motif-containing oligodeoxynucleotide (or CpG-ODN), e.g. which is capable of acting as a toll-like receptor 9 (TLR9) agonist. The CpG motif refers to an unmethylated cytidine-phospho-guanosine dinucleotide sequence, e.g. which is capable of binding to TLR9. Th1 response-directing adjuvants such as CpG promote the induction of a predominantly T helper type 1 (Th1) immune response in an immunized subject rather than a Th2 type response. In one embodiment, the CpG adjuvant comprised in the vaccine of the invention is a class A, class B or class C CpG (Campbell J D, 2017, in Christopher B. Fox (ed.), Vaccine Adjuvants: Methods and Protocols, Methods in Molecular Biology, vol. 1494, DOI 10.1007/978-1-4939-6445-1_2), preferably a class B CpG. Class B CpG molecules include CpG 1018 (TGACTGTGAACGTTCGAGATGA) (SEQ ID NO: 10), CpG 1826 (TCCATGACGTTCCTGACGTT) (SEQ ID NO: 11) and CpG 7909 (TCGTCGTTTTGTCGTTTTGTCGTT) (SEQ ID NO: 12). Most preferred is CpG 1018.

In an additional embodiment, combination of two or more different adjuvants is possible, especially if these adjuvants work synergistically or the combination of adjuvant induces both Th1 and Th2 immune responses. The Th1- or Th2-directing properties of commonly used vaccines are known in the art. One example of adjuvant combinations used in vaccines is the combination of CpG and alum, especially CpG 1018 and alum provided in the form of aluminium hydroxide (Al(OH)₃). Alum:CpG (w/w) ratio in the vaccine composition can be about 1:10, about 1:5, about 1:4, about 1:3, about 1:2, about 1:1, about 2:1, about 3:1, about 4:1, about 5:1, about 10:1, preferably between about 1:3 and 3:1, more preferably between about 1:2 and 1:1, most preferably about 1:2, even more preferably 1:2 in humans.

According to one aspect of the present invention, the composition comprising the lipidated Clostridium difficile toxin protein as defined above is useful for eliciting an immune response in a human subject.

According to another aspect, the composition of the present invention comprising the lipidated Clostridium difficile toxin protein is useful for the prevention or treatment of Clostridium difficile infection and/or Clostridium difficile-associated disease (CDAD) in a human subject in need thereof.

In particular, the treatment includes prevention of CDAD, protection from the infection, reducing or alleviating the symptoms of CDAD, or combinations thereof. Preferably, the composition of the present invention is for use in vaccination for preventing Clostridium difficile infection. Particularly, the composition of the present invention may be used to treat a subject at risk of CDAD, such as e.g. a subject with the following profile: i) a subject with a weaker immune system such as e.g. an elderly subject (e.g. a subject above 65 years of age) or a subject below 2 years of age; ii) an immunocompromised subject such as e.g. a subject with AIDS; iii) a subject taking or planning to take immunosuppressing drugs; iv) a subject with planned hospitalization or a subject that is in hospital; v) a subject in or expected to go to an intensive care unit (ICU); vi) a subject that is undergoing or is planning to undergo gastrointestinal surgery; vii) a subject that is in or planning to go to a long-term care such as a nursing home; viii) a subject with co-morbidities requiring frequent and/or prolonged antibiotic use; ix) a subject that is a subject with two or more of the above mentioned profiles, such as e.g. an elderly subject that is planning to undergo a gastrointestinal surgery; x) a subject with inflammatory bowel disease; and/or xi) a subject with recurrent CDAD such as e.g. a subject having experienced one or more episodes of CDAD.

The pharmaceutical compositions or vaccine according to the invention may be administered to a human subject as an injectable composition, for example as a sterile aqueous dispersion, preferably isotonic. The composition may be administered via a systemic or mucosal route. These administrations may include injection via the intramuscular, intraperitoneal, intradermal or subcutaneous routes; or via mucosal administration to the oral/alimentary, respiratory or genitourinary tracts. Although the vaccine of the invention may be administered as a single dose, components thereof may also be co-administered together at the same time.

In one embodiment, the pharmaceutical composition or vaccine according to the present invention described herein may be administered to a subject with, prior to, or after administration of one or more adjuvants.

Dosage schedule of administration and efficacy of the vaccine can be determined by methods known in the art. The amount of the vaccine and the immunization regimen may depend on the particular antigen and the adjuvant employed, the mode and frequency of administration, and the desired effect (e.g., protection and/or treatment). In general, the vaccine of the invention may be administered in amounts ranging between 1 g and 100 mg, such as e.g. between 60 μg and 600 μg. The pharmaceutical composition or vaccine according to the present invention may be administered to a human subject at a dose of from 20 to 200 μg. Necessity of administering one, two, three or more doses of the pharmaceutical composition (vaccine), as well as the immunization regimen can be determined by one skilled in the art by well-known methods. For example, a priming dose may be followed by 1, 2, 3 or more booster doses at weekly, bi-weekly or monthly intervals.

In a particular embodiment, the pharmaceutical composition or vaccine comprising the lipidated Clostridium difficile toxin B protein of SEQ ID NO: 6 (Lip-ToxB-His) and/or the Clostridium difficile toxin A protein of SEQ ID NO: 5 (Lip-ToxA-His) may be administered to a human subject at least one, two or three times at a total protein content of said 2 toxin proteins at a dose of from 20 to 200 μg.

In one embodiment, the population which can be treated according to the present invention includes healthy individuals who are at risk of exposure to C. difficile, especially, the individuals impending hospitalization or residence in a care facility, as well as personals in hospitals, nursing homes and other care facilities. In another embodiment, the population includes previously infected patients who relapsed after discontinuation of antibiotic treatment, or patients for whom antibiotic treatment is not efficient.

In one more embodiment of the invention, the population includes individuals who are at least 18 years or more of age. In one preferred embodiment, the human subject is from 18 to 65 years old. In another preferred embodiment, the human subject is elderly individuals over 65 years of age. The latter age group being the most vulnerable population suffering from C. difficile infections. In some more embodiment, the human subject is younger than 18 years of age.

Another aspect of the present invention is a method for the prevention or treatment of C. difficile infection and/or C. difficile-associated disease in a subject in need thereof, comprising administering to the subject an immunogenic composition comprising a lipidated C. difficile toxin B polypeptide, wherein the lipidated polypeptide comprises a C. difficile cell-binding domain or fragment thereof and wherein the lipidated polypeptide lacks the C. difficile toxin A cell-binding domain sequence.

In particular, according to the method of the present invention, the immunogenic composition is administered to the subject without administration of a C. difficile toxin A immunogenic polypeptide, preferably wherein the composition is administered to the subject as a sole immunogenic composition for the prevention or treatment of C. difficile infection and/or C. difficile-associated disease, wherein no further immunogenic composition for the prevention or treatment of C. difficile infection and/or C. difficile-associated disease is administered to the subject.

The terms “comprising”, “comprise” and “comprises” herein are intended by the inventors to be optionally substitutable with the terms “consisting of”, “consist of” and “consists of”, respectively, in every instance. The term “comprises” means “includes”. Thus, unless the context requires otherwise, the word “comprises”, and variations such as “comprise” and “comprising” will be understood to imply the inclusion of a stated compound or composition (e.g., nucleic acid, polypeptide, antibody) or step, or group of compounds or steps, but not to the exclusion of any other compounds, composition, steps, or groups thereof. The abbreviation, “e.g.” is derived from the Latin exempli gratia, and is used herein to indicate a non-limiting example. Thus, the abbreviation “e.g.” is synonymous with the term “for example”.

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a”, “an”, and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, may be approximate.

The present invention is further illustrated by the following Figures and Examples, from which further features, embodiments and advantages may be taken. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

### EXAMPLES

### Example 1. Lipidation Increases the Immunogenicity of C. difficile Toxin A and Toxin B Proteins

Immunization of mice Female C57BL/6N mice were used for all studies (Janvier, France). Prior to the first immunization mice were bled and pre-immune sera were prepared. Eight antigens, lipidated and or non-lipidated were tested alone or in combinations. Mice were immunized intraperitoneally (200 μL) twice with two weeks interval. Doses used were either 5 or 10 μg of the respective antigen, all vaccines were formulated with aluminium hydroxide at a final concentration of 0.15%. Two weeks after the second immunization blood was collected and sera were prepared. Each group consists of ten mice, one group were injected with PBS formulated with aluminium hydroxide and served as negative control.

Immunogenicity with ELISA Immune sera derived two weeks after the second immunization were analyzed for C-TAB.G5.1, toxin A and toxin B specific IgG titers. Indirect ELISA were performed using C-TAB.G5.1, toxoid A and toxoid B as coating antigens. Microtiter plates were coated with antigen (C-TAB.G5.1, toxoid A or toxoid B) diluted to 1.0 μg/mL in PBS (100 μL/well). Following overnight incubation at 2-8° C., plates were washed, blocking buffer was added (200 μL/well) and the plates incubated for 2 hours at room temperature. Next, plates were washed, and dilutions of mouse sera prepared in ELISA diluent buffer, were added to plates in duplicate wells. Starting at a 1:100 dilution in the first well, seven 4-fold serial dilutions were prepared. The following day, plates were washed and 100 μL of a peroxidase-conjugated anti-mouse IgG was added. Following a 2 hours incubation at room temperature, plates were washed and 100 μL ABTS [2,2′-azino-di(3-ethylbenzthiazoline-6-sulfonate)] were added to all wells. Cleavage of the ABTS substrate by the peroxidase resulted in the development of a blue-green reactant. Following a 30 minute incubation at room temperature enzymatic conversion was stopped by adding 50 μL of a 1% SDS stop solution. Absorbance at 405 nm was measured in a plate reader. The data was analyzed using the four-parameter logistic fit equation. ELISA results are given in ELISA Units (EU) which represents the inverse of the sample dilution resulting in an A405 of 0.5.

Immunogenicity with Toxin Neutralization Assay (TNA)

The generation of functional antibodies was determined in a toxin neutralization assay (TNA) with T84 cells. T84 cells were seeded at 1×10⁵ cells/well in 96-well plates and grown for approximately 28 hours at 37° C., 5% CO₂ (cell culture medium: DMEM/F-12 with 2.5 mM Glutamine, HEPES and Phenol red supplemented with 5% FBS and Penicillin/Streptomycin). On the following day, serial 4-fold dilutions of mouse sera (eight dilutions from 1:10 to 1:163840 in serum-free assay medium) were incubated for one hour with an equal volume of C. difficile toxins (final minimum serum dilution 1:20). Toxin A or toxin B was used at a final concentration of 4×EC₅₀ as determined in toxin titration experiments. The mixture was subsequently added in duplicate to a monolayer of T84 cells. Cells were incubated for further 42-43 hours at 37° C., 5% CO₂ before 0.02% Neutral Red solution was added. Subsequently, cells were washed several times in order to remove excess staining solution and toxin-affected cells that lost adherence. After addition of extraction solution (1% acetic acid in 50% ethanol) the absorbance of released Neutral Red was measured. Absorbance of Neutral Red was quantified at 542 nm. Results were calculated using analysis software SoftMax Pro 5.2 G×P. Curves were created by applying a four-parameter logistic curve fit. The toxin neutralizing titer was determined as the inverse value of the final serum dilution which caused 50% protection of T84 cells from toxin-induced cell rounding/loss of adherence. Sample curves were constrained to the upper and the lower asymptote of the reference substance curve (parameter A and D), to allow reliable titer calculation. The titer of toxin neutralizing antibodies present in the serum sample corresponds to the EC50 (parameter C) of the four-parameter curve fit. Samples with an EC50<20 (lowest sample dilution tested 1:20 does not reach a 50% protection) were rated negative.

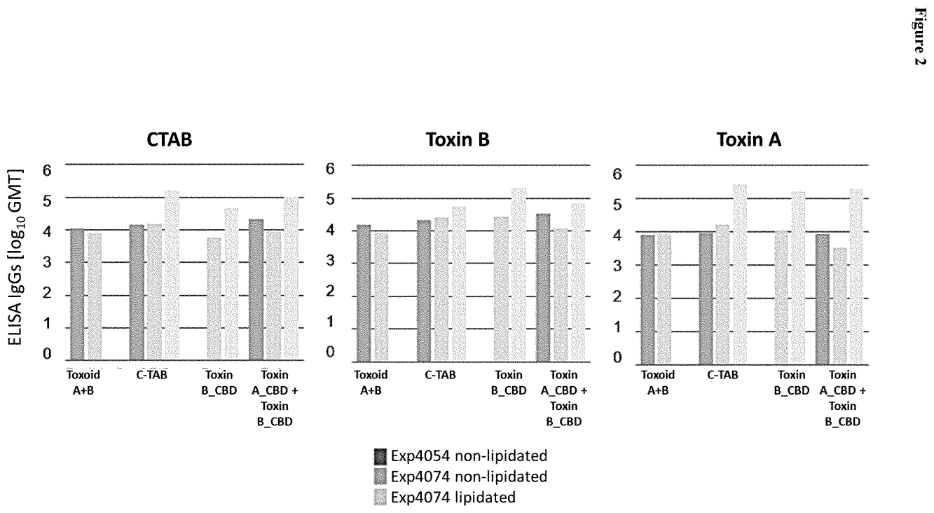

FIG. 2 shows titers of anti-Toxin A or anti-Toxin B serum antibodies (bulk IgGs) elicited in mice immunized with the lipidated or non-lipidated polypeptides containing C. difficile Toxin A and/or Toxin B cell-binding domains (CBD), particularly Toxin A_CBD (SEQ ID NO: 5), Toxin B_CBD (SEQ ID NO: 6) and CTAB fusion (SEQ ID NO: 7). As the result of this experiment, antibody titers raised against lipidated forms of both Toxin A_CBD and toxin B-CBD proteins, as well as CTAB fusion protein are higher than titers raised against non-lipidated forms of these proteins.

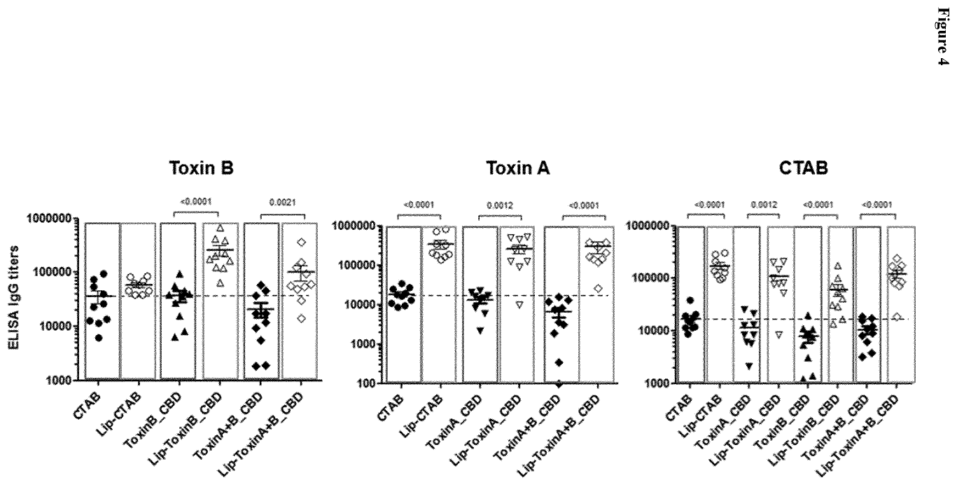

Also a statistically significant increase of specific antibody titers raised against lipidated protein constructs containing Toxin A or Toxin B cell-binding domain sequences as compare to the same non-lipidated constructs is demonstrated in FIG. 4.

At the same time, anti-Toxin B antibody titers induced in the presence of the lipidated protein containing a Toxin A_CBD sequence are lower compared to titers raised against the lipidated protein just containing Toxin B_CBD. This phenomenon is not true for lipidated Toxin A_CBD containing proteins.

### Example 2. Mice Protection Upon Immunization with the Lipidated C. difficile Toxin B Protein

### Immunization and Challenge of Mice

Female C57BL/6N mice were immunized as described in Example 1. Two weeks after the second immunization blood was collected and sera were prepared. Three weeks after the second immunization mice were challenged with a lethal dose of Clostridium difficile toxin B from strain VPI10463 (Native Antigen, UK). Mice were injected intraperitoneally (100 μL) with C. difficile toxin B, survival was monitored for 14 days.

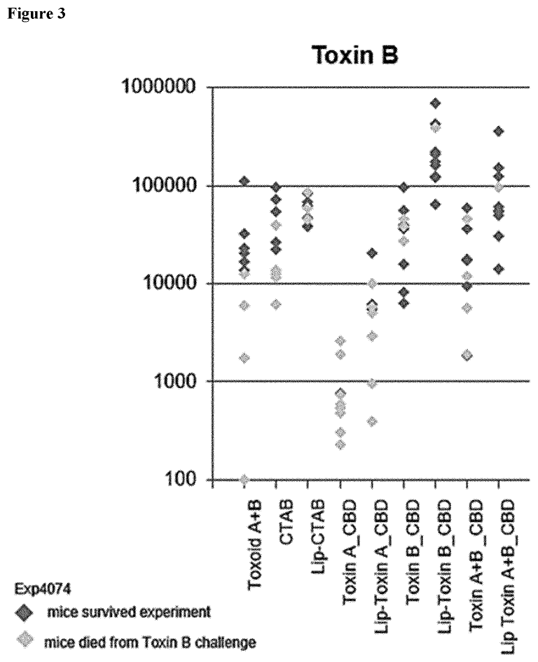

The result of this study is shown in FIG. 3. This result demonstrates that higher anti-toxin B antibody titer supports survival of mice upon Toxin B challenge and lower titers lead to death. Particularly, only those mice that have anti-toxin B IgG titer>100,000 are fully protected (with one exception) against Toxin B challenge. The highest level of specific antibodies is elicited upon immunization with the lipidated Toxin B_CBD protein (SEQ ID NO: 6).

### Further Aspects of the Invention:

1.An immunogenic composition for use in the prevention or treatment of C. difficile infection and/or C. difficile-associated disease in a subject, the composition comprising:
a lipidated C. difficile toxin B polypeptide, wherein the lipidated polypeptide comprises (i) a C. difficile toxin B cell-binding domain or fragment thereof and (ii) lacks a C. difficile toxin A cell-binding domain sequence.
2. The immunogenic composition of aspect 1, wherein the lipidated C. difficile toxin B polypeptide is covalently linked to a lipid moiety.
3. The immunogenic composition of aspect 1, wherein the C. difficile toxin B cell-binding domain or fragment thereof is capable of binding to a host cell receptor.
4. The immunogenic composition of aspect 1, wherein the composition further comprises a C. difficile antigen.
5. The immunogenic composition of aspect 4, wherein the C. difficile antigen is selected from the group consisting of a C. difficile toxin A polypeptide, a C. difficile surface layer protein, and a C. difficile flagellar protein.
6. The immunogenic composition of aspect 1, wherein the lipidated C. difficile toxin B polypeptide is formulated with an adjuvant.
7. The immunogenic composition of aspect 6, wherein the adjuvant is selected from the group consisting of an aluminum salt, a saponin, an oil-in-water emulsion, and a toll-like receptor agonist.
8. A method for preventing or treating C. difficile infection and/or C. difficile-associated disease in a subject, the method comprising administering to the subject an effective amount of the immunogenic composition of aspect 1.
9. The method of aspect 8, wherein the subject is at risk for developing C. difficile infection and/or C. difficile-associated disease.
10. The method of aspect 8, wherein the subject has been diagnosed with C. difficile infection and/or C. difficile-associated disease.
11. The method of aspect 8, wherein the administering is performed via a route selected from the group consisting of intramuscular, subcutaneous, intradermal, and mucosal administration.
12. The method of aspect 8, wherein the administering comprises a single dose or a plurality of doses.
13. The method of aspect 12, wherein the plurality of doses are administered at predetermined intervals.
14. The method of aspect 8, wherein the effective amount of the immunogenic composition is determined based on the subject's age, weight, and health status.
15. The method of aspect 8, further comprising monitoring the subject for a reduction in the incidence or severity of C. difficile infection and/or C. difficile-associated disease.
16. A kit for preventing or treating C. difficile infection and/or C. difficile-associated disease in a subject, the kit comprising:
   a) the immunogenic composition of aspect 1; and
   b) instructions for administering the immunogenic composition to the subject.

The invention claimed is:

1. An immunogenic composition comprising a lipidated C. difficile toxin B polypeptide for use in the prevention or treatment of C. difficile infection and/or C. difficile-associated disease in a subject, wherein the lipidated polypeptide comprises (i) a C. difficile toxin B cell-binding domain or fragment thereof and (ii) lacks a C. difficile toxin A cell-binding domain sequence, and wherein the composition (iii) optionally comprises a further C. difficile antigen.

2. An immunogenic composition for use according to claim 1, wherein the immunogenic composition does not comprise a C. difficile toxin A C-terminal repeat domain-containing polypeptide.

3. An immunogenic composition for use according to claim 1 or claim 2, wherein the lipidated polypeptide is the sole active agent in the immunogenic composition.

4. An immunogenic composition for use according to any preceding claim, wherein the composition is administered to the subject without administration of a C. difficile toxin A immunogenic polypeptide, preferably wherein the composition is administered to the subject as a sole immunogenic composition for the prevention or treatment of C. difficile infection and/or C. difficile-associated disease.

5. An immunogenic composition for use according to claim 1, wherein the immunogenic composition further comprises a lipidated C. difficile toxin A polypeptide comprising a toxin A cell-binding domain or fragment thereof.

6. An immunogenic composition for use according to claim 1, wherein the immunogenic composition is administered to the subject in combination with a further immunogenic composition comprising a lipidated C. difficile toxin A polypeptide comprising a toxin A cell-binding domain or fragment thereof, preferably wherein the lipidated C. difficile toxin A polypeptide lacks C. difficile toxin B cell-binding domain or C-terminal repeat domain sequence.

7. An immunogenic composition for use according to any preceding claim, wherein the lipidated C. difficile toxin B polypeptide comprises or consists of a lipidated form of SEQ ID NO: 6, or a lipidated form of the full length C. difficile toxin B C-terminal repeat domain sequence of SEQ ID NO: 4, or a variant thereof with a sequence identity to SEQ ID Nos 4 or 6 of at least about 80%, preferably about 90%, more preferably about 95%.

8. An immunogenic composition for use according to any of claims 5 to 7, wherein the lipidated C. difficile toxin A polypeptide comprises or consists of a lipidated form of SEQ ID NO: 5, or a lipidated form of the full length C. difficile toxin A C-terminal repeat domain sequence of SEQ ID NO: 3, or a variant thereof with a sequence identity to SEQ ID Nos 3 or 5 of at least about 80%, preferably about 90%, more preferably about 95%.

9. An immunogenic composition for use according to any preceding claim, wherein the composition further comprises a C. difficile antigen selected from the group consisting of Acd protein (WP_009892971.1), C40 family peptidase (WP_009890599.1), Cwp66, Cwp84, FliC, FliD, CDTa, CDTb, Bc1A3 glycoprotein, PSII antigen and rlipoA-RBD.

10. An immunogenic composition for use according to any preceding claim, wherein the composition further comprises a pharmaceutically acceptable carrier and/or excipient, and optionally one or more adjuvant(s).

11. An immunogenic composition for use according to claim 10, wherein the composition comprises a CpG-containing oligodeoxynucleotide (CpG-ODN) and/or an alum adjuvant.

12. An immunogenic composition for use according to claim 11, wherein the CpG-ODN is CpG1018 as defined by SEQ ID NO: 10.

13. An immunogenic composition for use according to claim 11, wherein the alum adjuvant is aluminium hydroxide comprising less than 1.25 ppb copper based on the weight of the composition.

14. A method for the prevention or treatment of C. difficile infection and/or C. difficile-associated disease in a subject in need thereof, comprising administering to the subject an immunogenic composition comprising a lipidated C. difficile toxin B polypeptide, wherein the lipidated polypeptide comprises a C. difficile cell-binding domain or fragment thereof and wherein the lipidated polypeptide lacks the C. difficile toxin A cell-binding domain sequence.

15. The method according to claim 14, wherein the immunogenic composition is administered to the subject without administration of a C. difficile toxin A immunogenic polypeptide.

16. The method according to claim 14, wherein the immunogenic composition is administered to the subject without administration of a C. difficile toxin A immunogenic polypeptide, preferably wherein the composition is administered to the subject as a sole immunogenic composition for the prevention or treatment of C. difficile infection and/or C. difficile-associated disease, wherein no further immunogenic composition for the prevention or treatment of C. difficile infection and/or C. difficile-associated disease is administered to the subject.
